(19) United States
(12) Patent Application Publication
(10) Pub. No.: US 2006/0040264 A1
(43) Pub. Date: Feb. 23, 2006

(54) Means for identifying neisseria meningitidis-specific genes

(76) Inventors: Xavier Nassif, Paris (FR); Vladimir Pelicic, Paris (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/500,553

(22) PCT Filed: Dec. 30, 2002
(86) PCT No.: PCT/FR02/04587

§ 371 Date: Feb. 24, 2005

(30) Foreign Application Priority Data
Dec. 31, 2001 (FR) 01 17 088
Apr. 3, 2002 (FR) 02 04 166

(51) Int. Cl. C12Q 1/68; C12Q 1/04

(52) U.S. Cl. 435/6; 435/34

(57) ABSTRACT

The invention concerns an exhaustive method for detecting pathogenic bacteria genes, in particular Nm genes, expressing a desired phenotype, characterized in that it consists in: using a bank of mutants generated from given bacterial strain so that at least 70% of the non-essential genes, and in particular at least 80%, even more than 90%, are mutagenized by inserting a transposon in a reading phase; then contacting the mutants, either individually, or in groups, with an environment, such as a medium, an animal or cells, capable of interacting with the mutant bacteria expressing the desired phenotype; recovering, when groups are used, the bacteria which have not reacted with the desired phenotype; identifying the mutated genes of said bacteria and verifying whether they are involved in said phenotype. The invention is useful, in particular, as anti-pathogenicity targets, which consists in inhibiting Neisseria meningitidis growth in vivo in the serum, for developing antibiotics, for screening and manufacturing medicines designed to open the blood brain barrier to active principles, and for preparing vaccines.

The invention relates to means for identifying genes specific to Neisseria meningitidis (Nm in abstract). It also relates to these genes and their biological applications.

Nm is a strictly human bacteria which does not survive in the external environment. It's only known reservoir is the nasopharynx of humans. In certain circumstances which are still little understood, this bacteria will leave the nasopharynx, infiltrate the blood in circulation and cause septicaemia and/or meningitides. The existence of a meningitis suggests that the bacteria crosses the blood-brain barrier, one of the most difficult barriers to cross in the organism. Neisseria meningitidis is a bacteria having extracellular multiplication, in other words its dissemination in vivo is accompanied by a multiplication in the interstitial area. Very few bacteria having extracellular multiplication are capable of crossing the blood-brain barrier after the neonatal period, they are essentially Streptococcus pneumoniae, Haemophilus influenzae and Neisseria meningitidis. This property thus suggests specific attributes which allow these microorganisms to cross this barrier.

Neisseria meningitidis presents two specificities for a bacteria having extracellular multiplication:

(i) It is responsible for substantial bacteremia with a high number of bacteria in the blood. Thus, the comparison, in an animal model using the new-born rat, of the level of bacteremia induced by the injection of the same number of bacteria belonging to two different species (Neisseria meningitidis and Klebsiella pneumoniae) shows that N. meningitidis induces a bacteremia which can be 50-100 times greater than that induced by K. pneumoniae. This underlines the perfect adaptation of N. meningitidis to growth in the extra-cellular area. Certain bacterial attributes have already been identified as participating in this extracellular growth. These are essentially the polysaccharidic capsule, the lipooligosaccharide and the iron capture systems. The two first attributes allow resistance to the complement and to phagocytosis by the granulocytes and the third attribute allows the bacteria to obtain the iron essential for its growth.

(ii) The second particularity of N. meningitidis is related to its ability to cross the blood-brain barrier. This property results from an interaction with the cerebral endothelial cells. Until now the only bacterial attribute identified as being involved in the interaction of N. meningitidis at the cerebral endothelium level are the type IV pili. A molecule which is one of these pili called PilC, involved in this interaction, is the adhesin of the pili.

The inventors work has concerned the search for means allowing identification of the genes of Nm which are capable of growing specifically in serum and of crossing the blood-brain barrier.

The application to Nm of the technique described by Pelicic et al, 2000 for building a bank of mutants allowed mutagenization of more than 70% of the mutagenizable and thus non essential genes.

This tool has proved to be particularly valuable for detecting in an exhaustive fashion all of the mutants for a given phenotype, for example those which are important for growth in the serum, and for identifying adhesins which are important for interaction with the endothelial cells and thus the crossing of the blood-brain barrier and this is without necessarily testing the mutants individually for this phenotype.

Therefore the invention relates to the use of such a bank for detecting genes of Nm expressing a particular phenotype.

It also relates to the genes involved in such a phenotype.

The invention also relates to the exploitation of the thus-identified genes as Nm anti-pathogenicity targets.

It also relates to the use of the genes coding for adhesins to allow therapeutic ingredient to pass through the blood-brain barrier.

The invention moreover relates to the essential genes of N. meningitidis, and their homologues in other bacterial species and their use as targets for developing antibiotics.

According to the invention, genes of pathogenic bacteria, in particular of Nm, are detected, expressing a desired phenotype, according to a method characterized in that:
a bank of mutants generated from a given bacterial strain is used so that at least 70% of the non-essential genes, and in particular 80 %, or even more than 90%, are mutagenized by inserting a transposon in a reading frame,
the mutants are then brought into contact, either individually, or in pools, with an environment, such as a medium, an animal or cells, capable of interacting with the mutant bacteria expressing the desired phenotype,
when pools are used, the bacteria which have not reacted with the desired phenotype are recovered,
the mutated genes of these bacteria are identified and their involvement in said phenotype is verified.

The bank of mutants is advantageously generated according to the method described by Pelicic et al. above.

The contact stage is carried out by passing on serum or an animal model in vivo or cells which are able to react with the bacteria expressing the desired phenotype and, when pools of mutants are used, the bacteria which have not reacted with the desired phenotype are recovered.

In order to identify the mutated genes of these bacteria and to verify their involvement in said phenotype, the mutants are organized into pools. For each mutant, the insertion sites are amplified using appropriate oligonucleotides. The amplification products are placed on a membrane made for example of nylon. The pools of mutants are placed under the conditions for which mutants are sought. Total DNA is prepared using bacteria obtained from each output pool and an amplification is carried out using oligonucleotides which served to amplify the insertion sites in the mutants of the pool. The amplification product then serves to hybridize the membranes which correspond to each pool. The mutants for which no amplification is detected are mutants for the phenotype considered. It will be observed that this technique allows the mutants in question to be retained, allowing each mutation to be retransformed in order to confirm the phenotype.

The invention also relates to the genes which give a bacteria the ability to grow or to react with a given environment such as serum, an animal model in vivo, cells.

These genes are characterized in that they can be obtained by the method defined above.

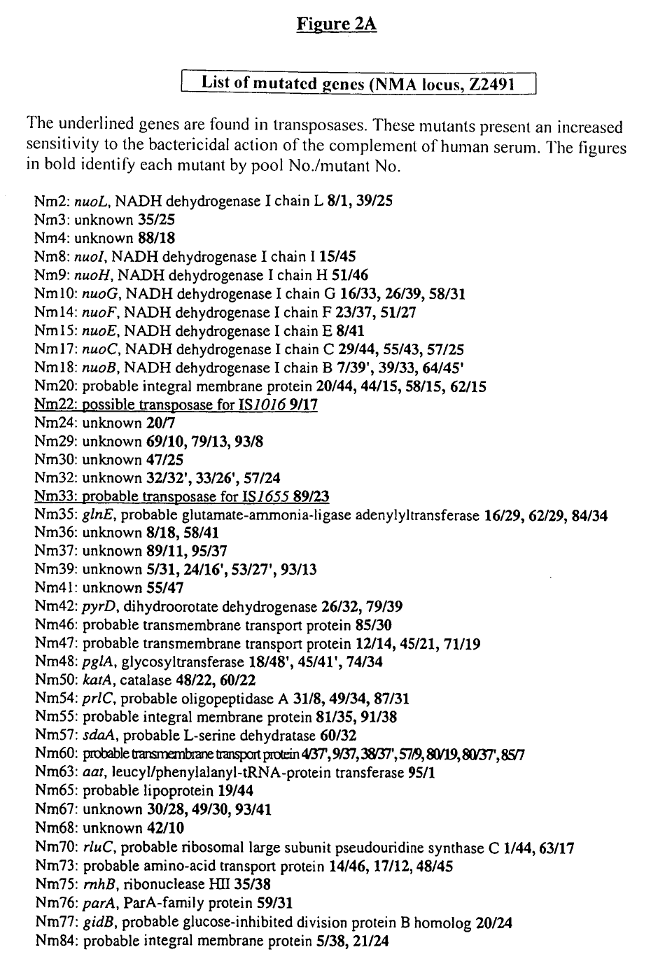

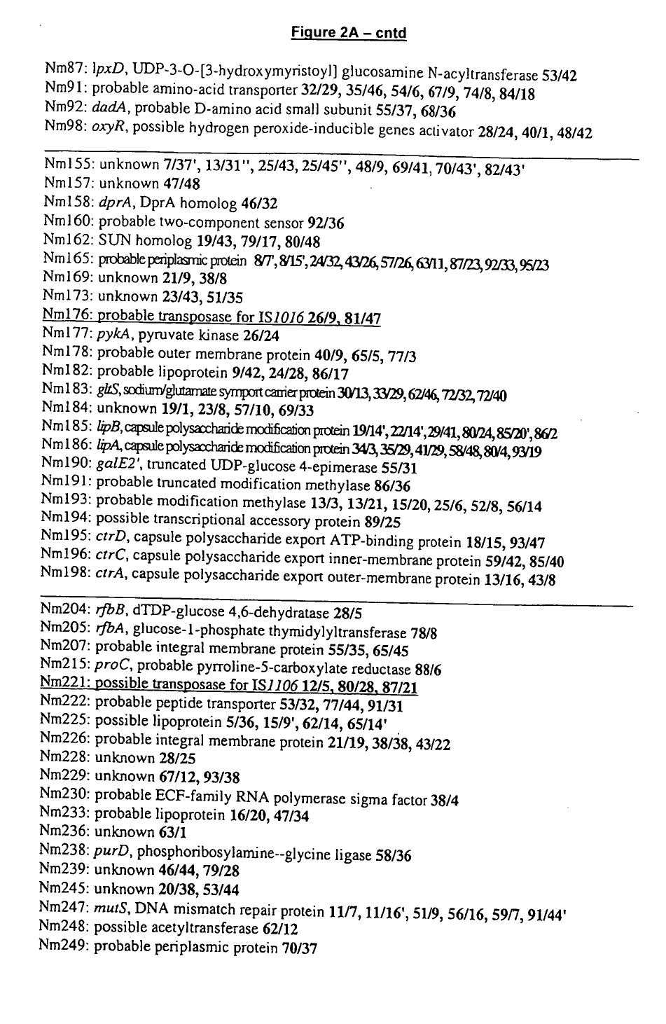

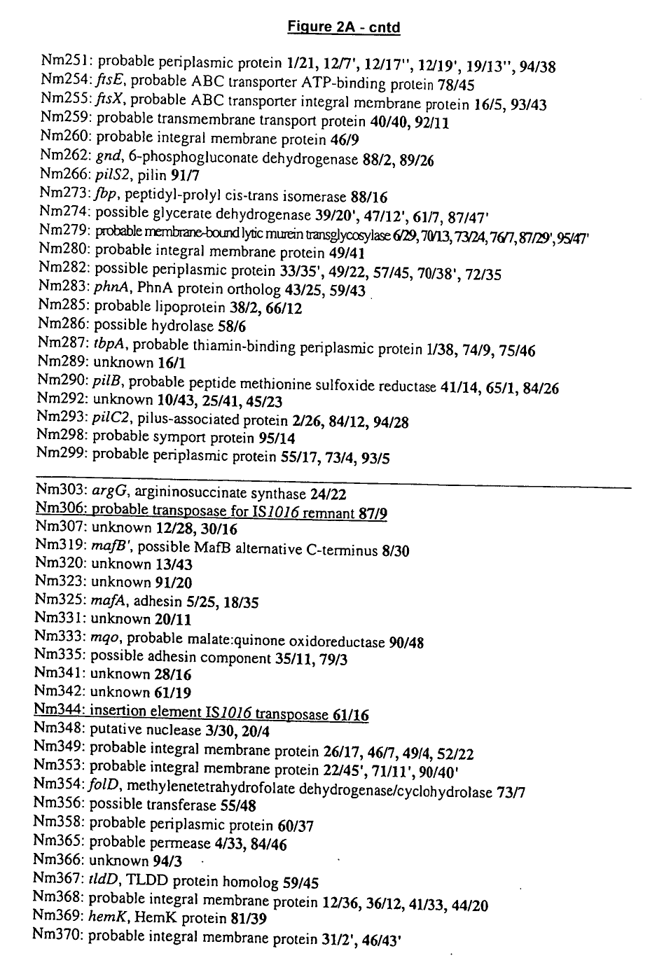

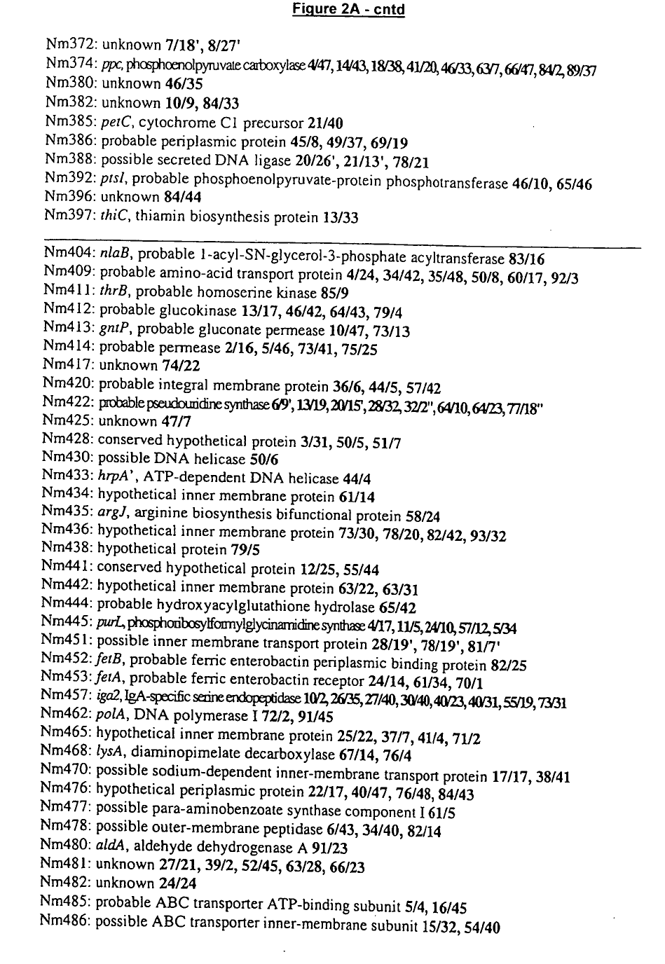

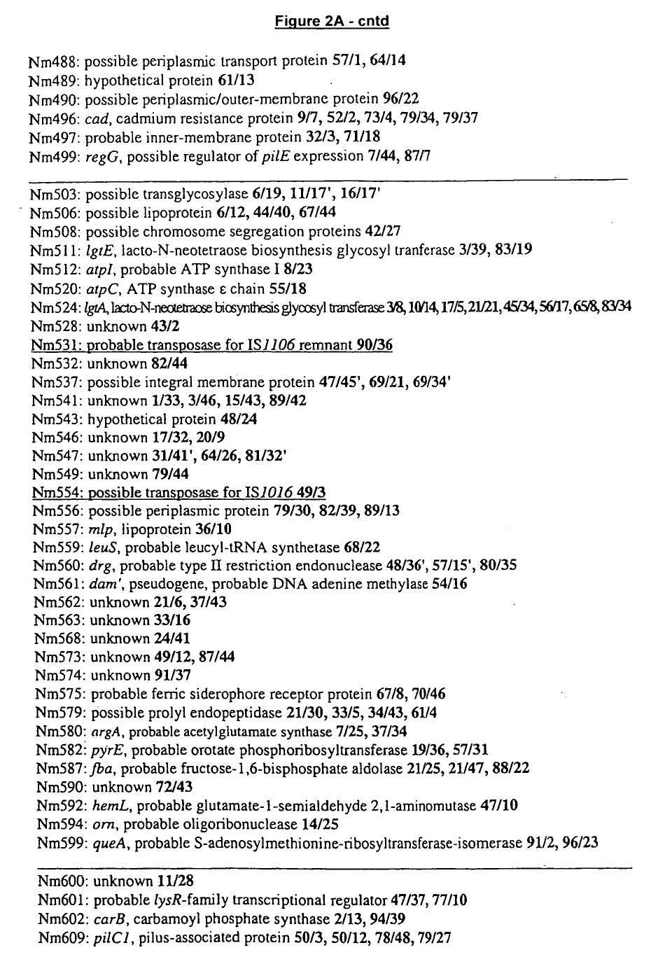

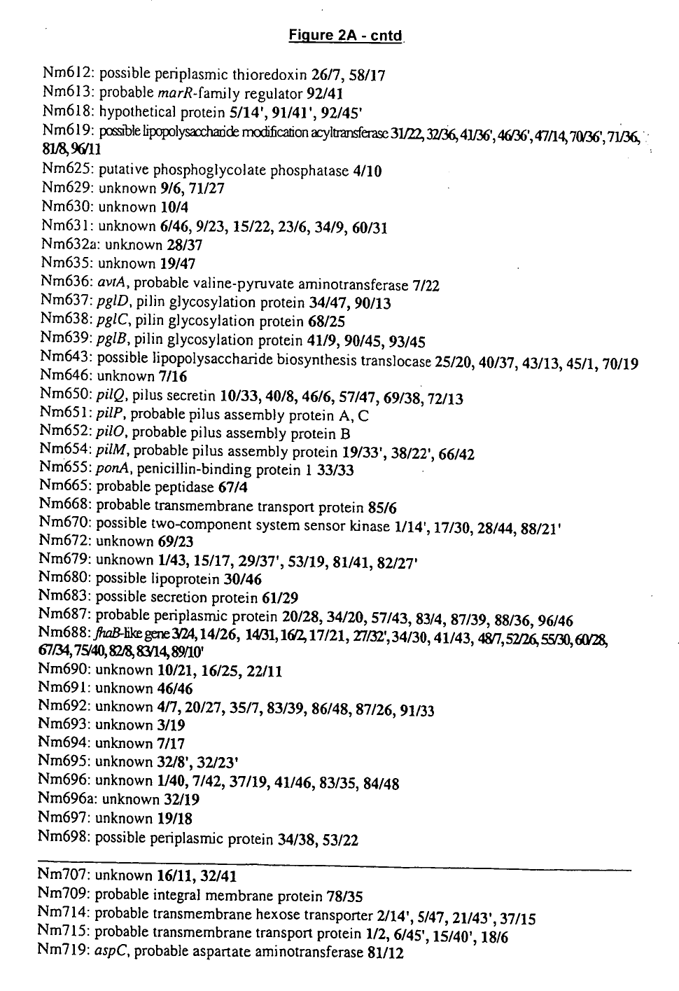

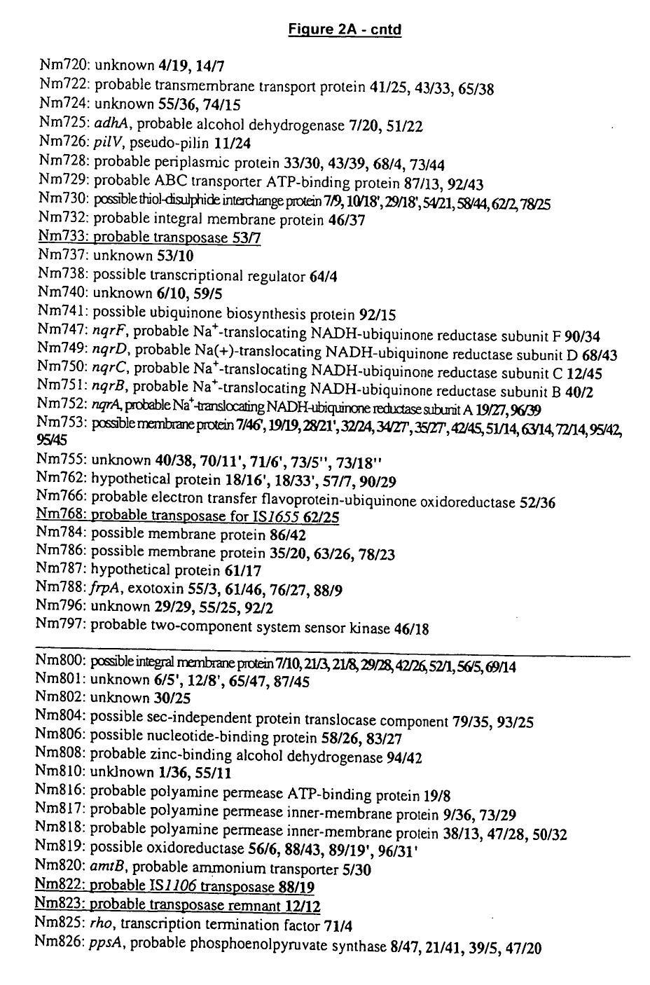

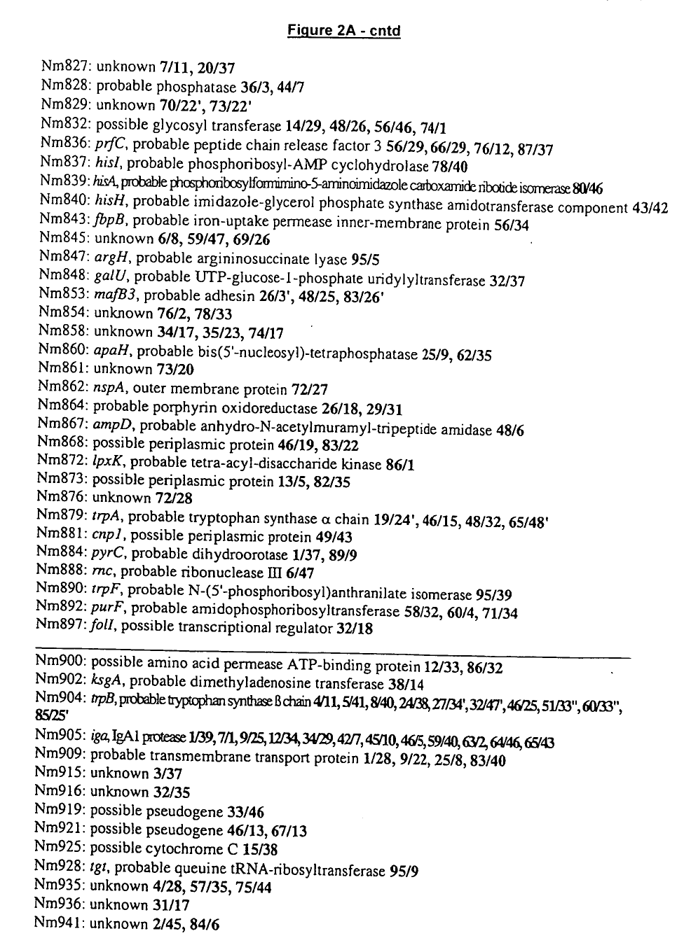

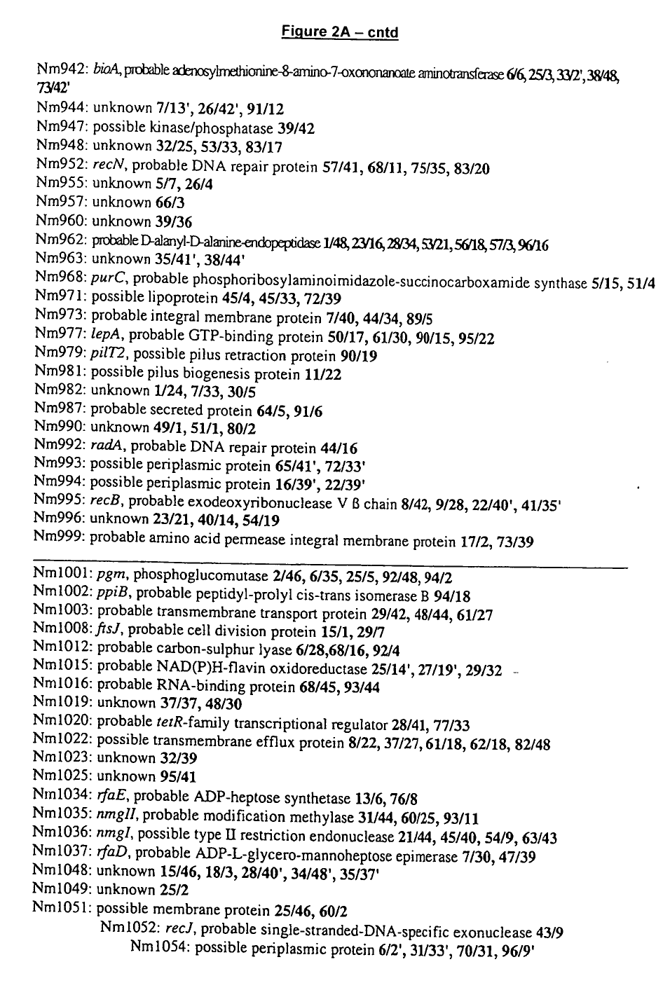

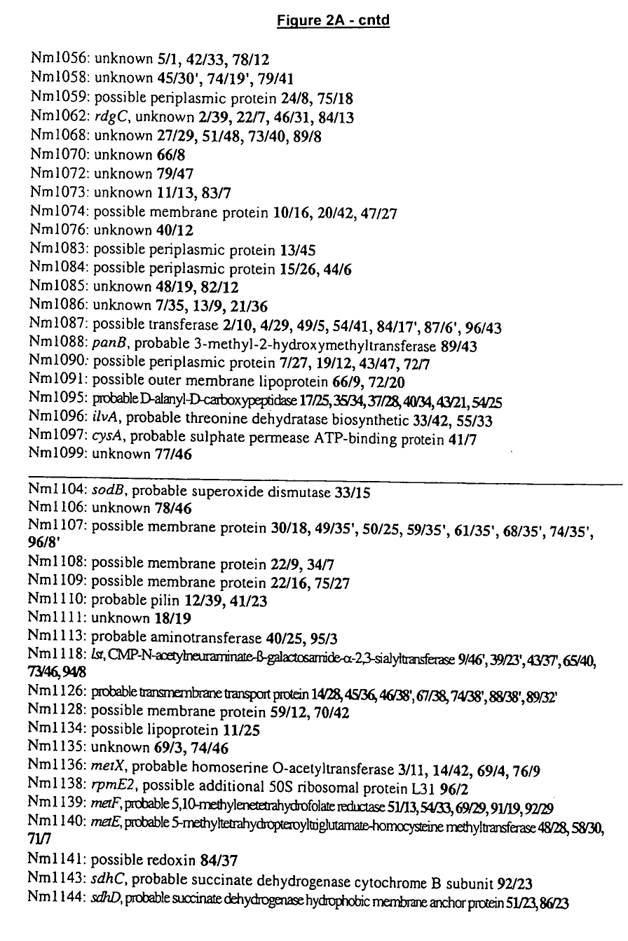

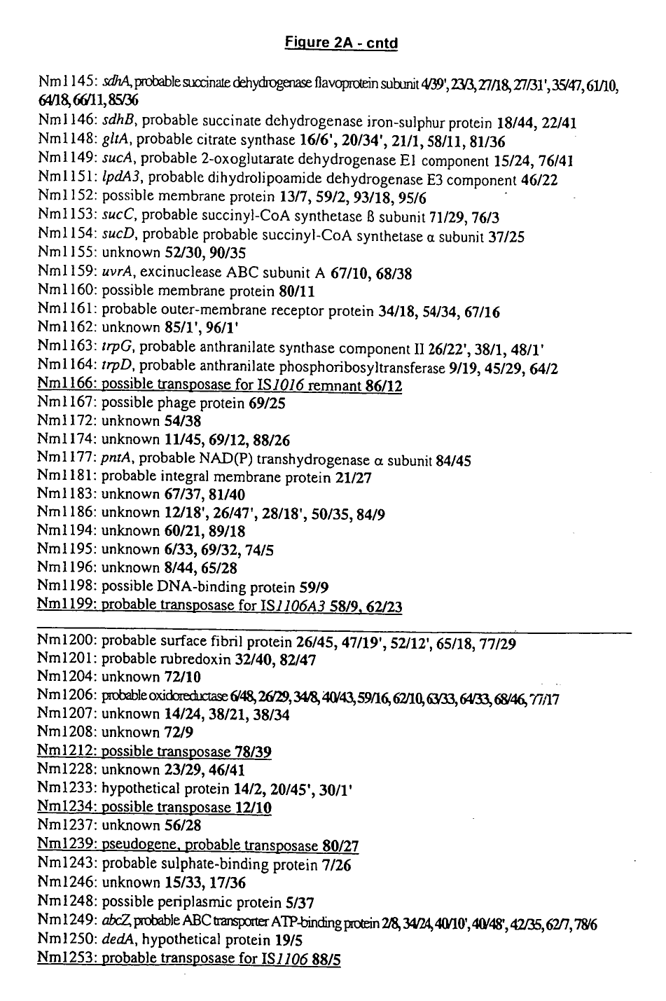

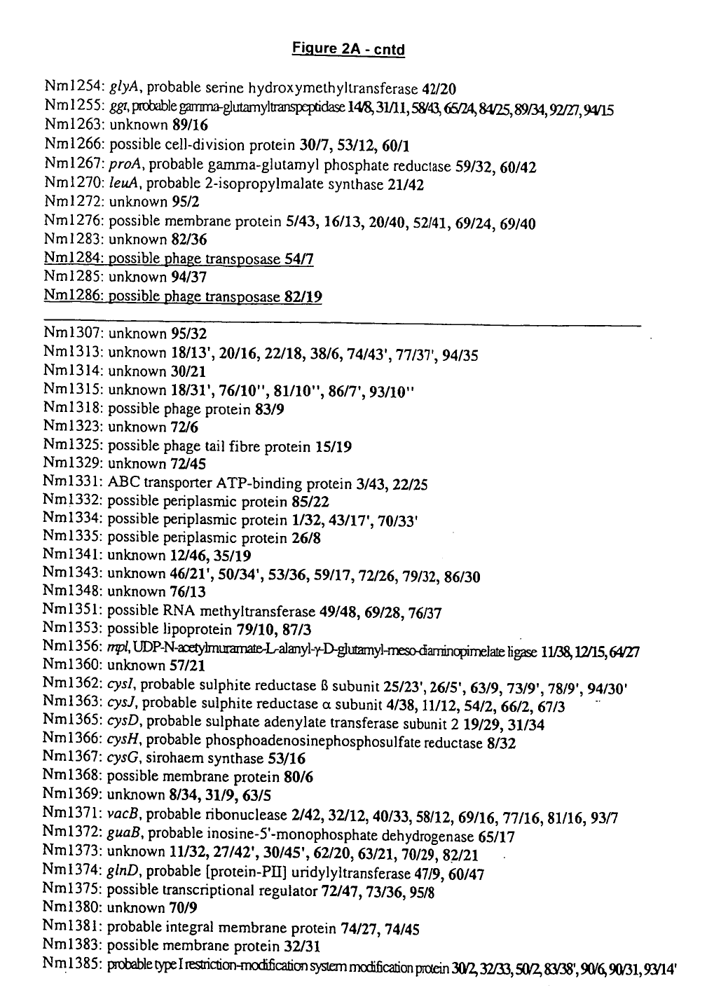

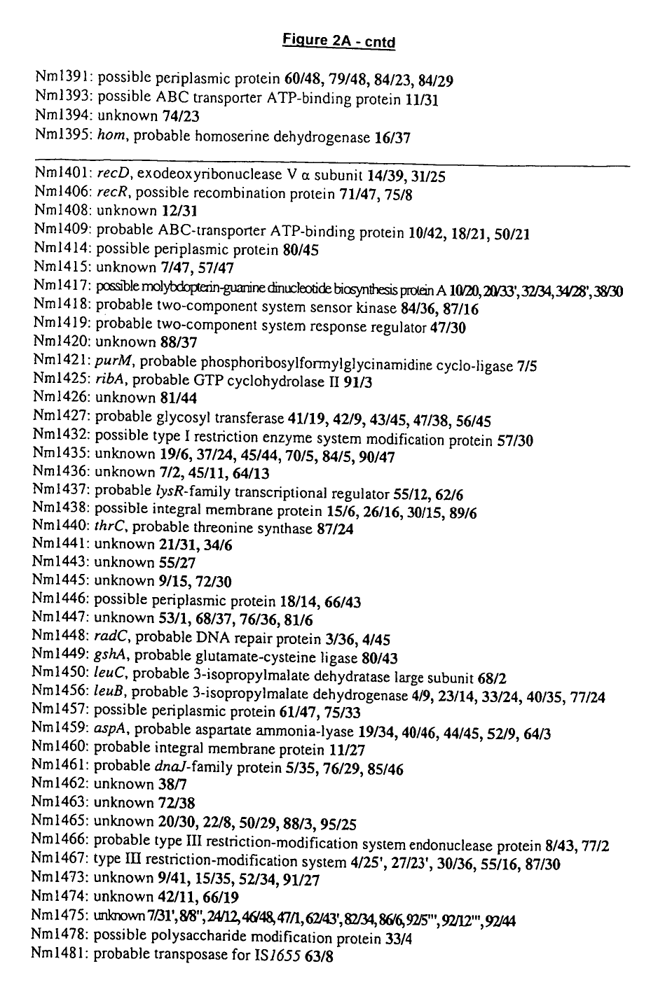

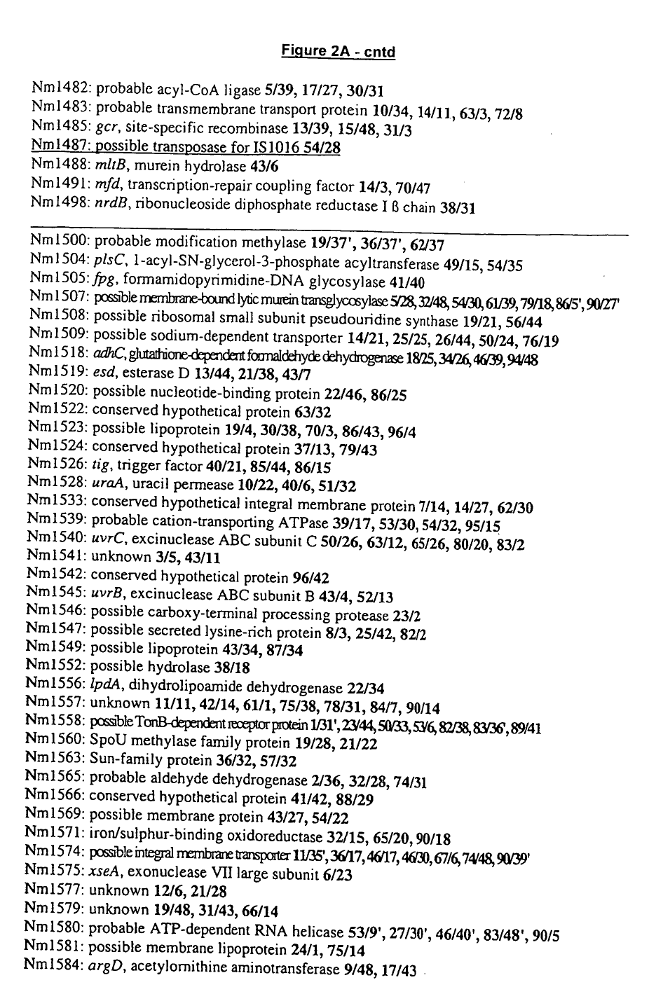

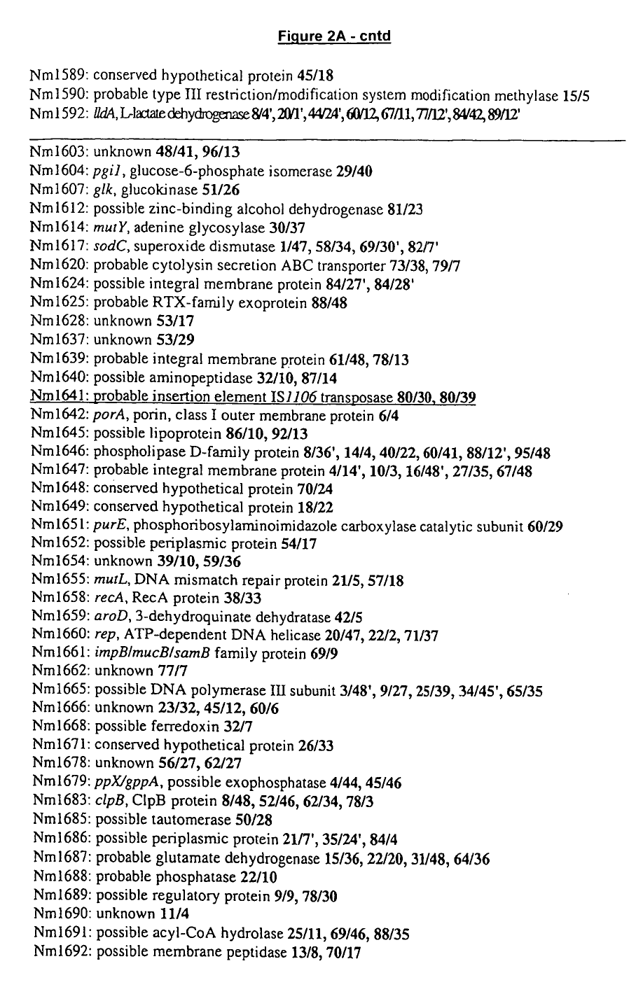

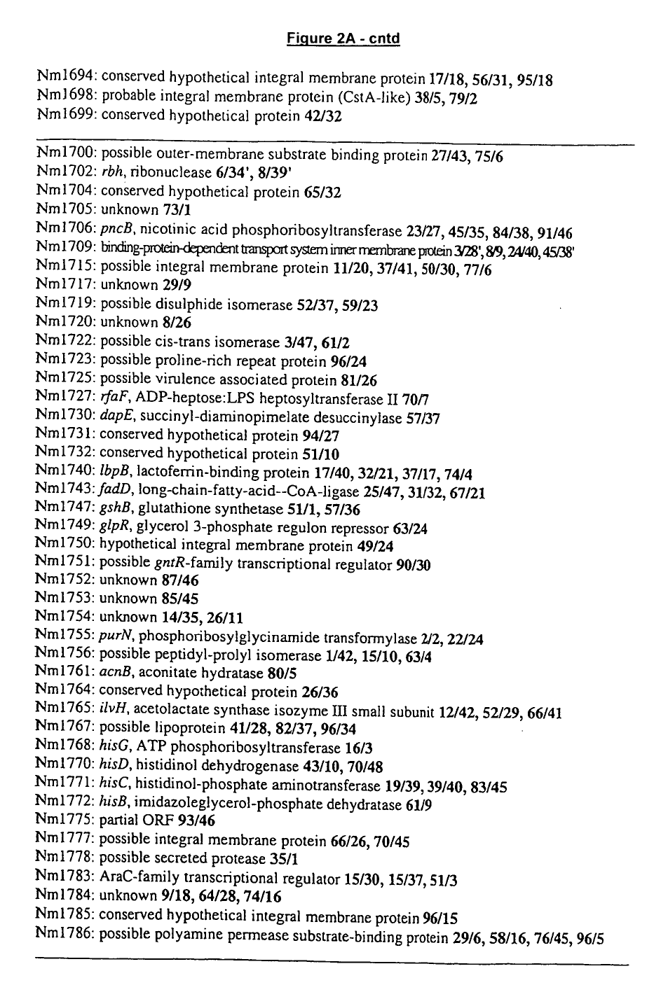

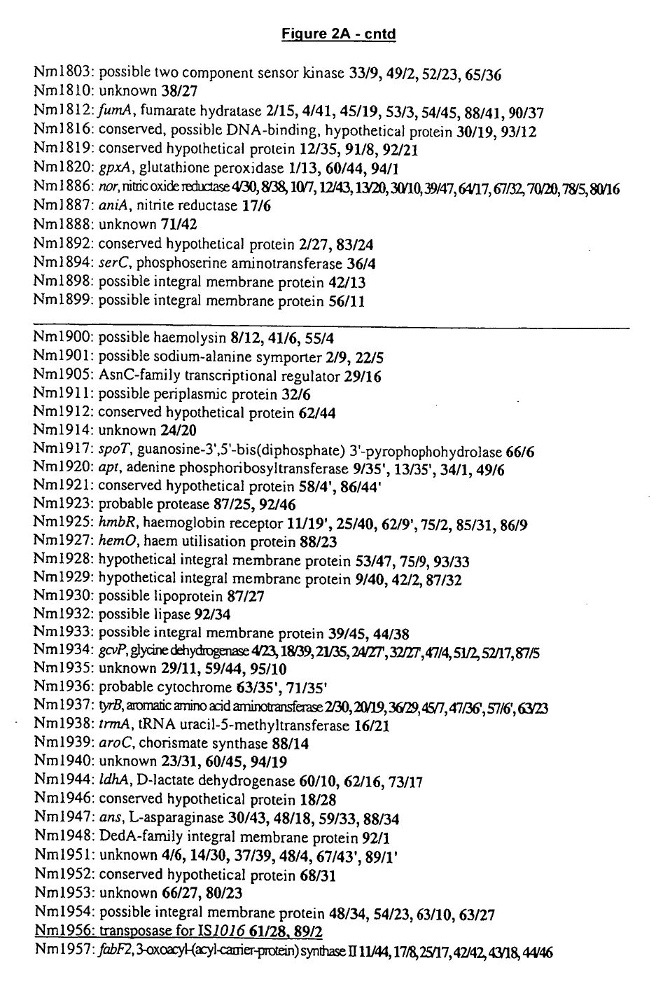

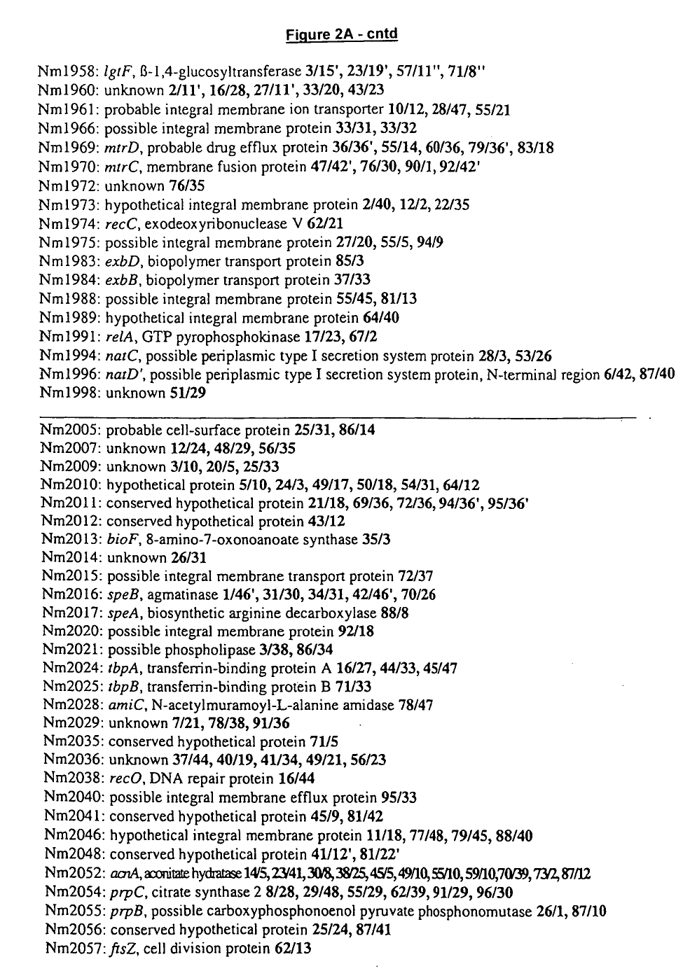

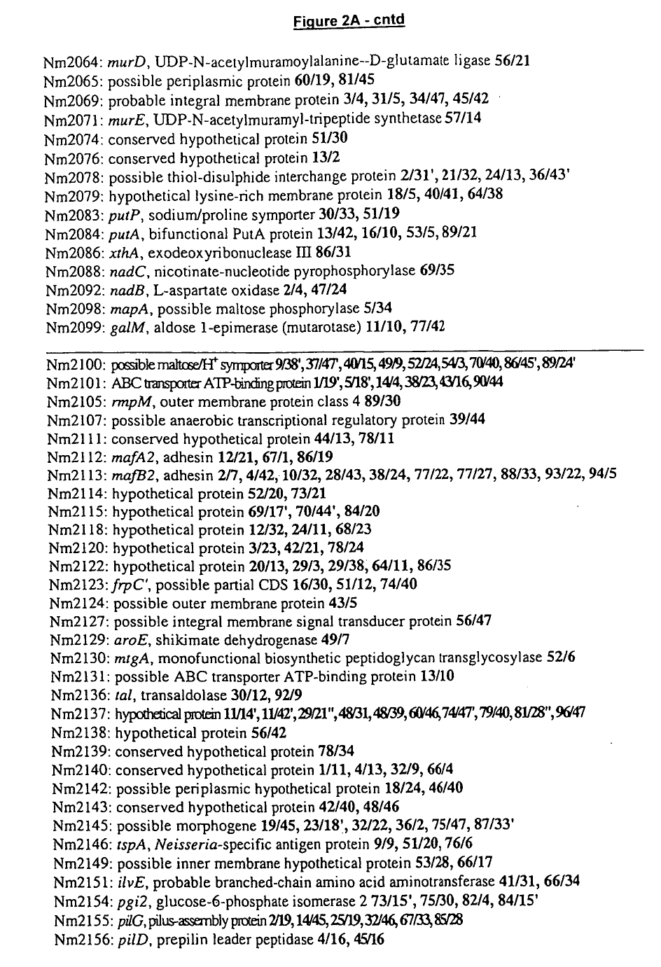

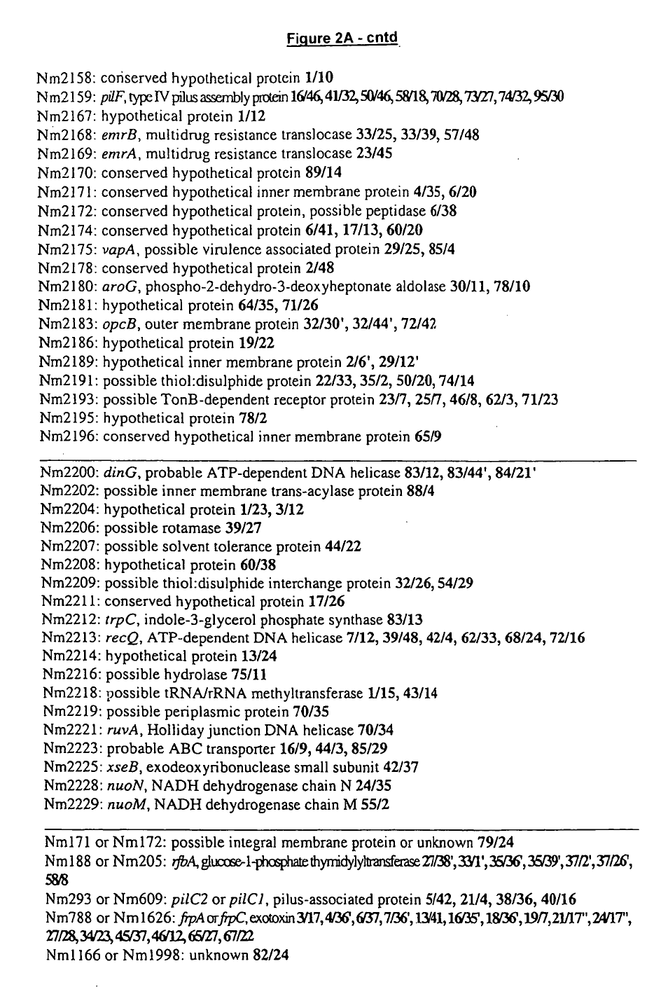

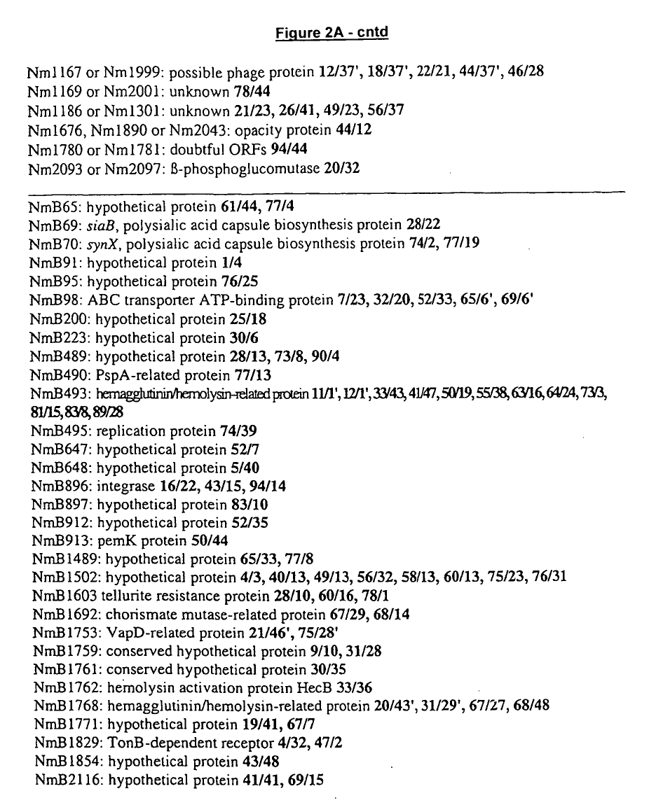

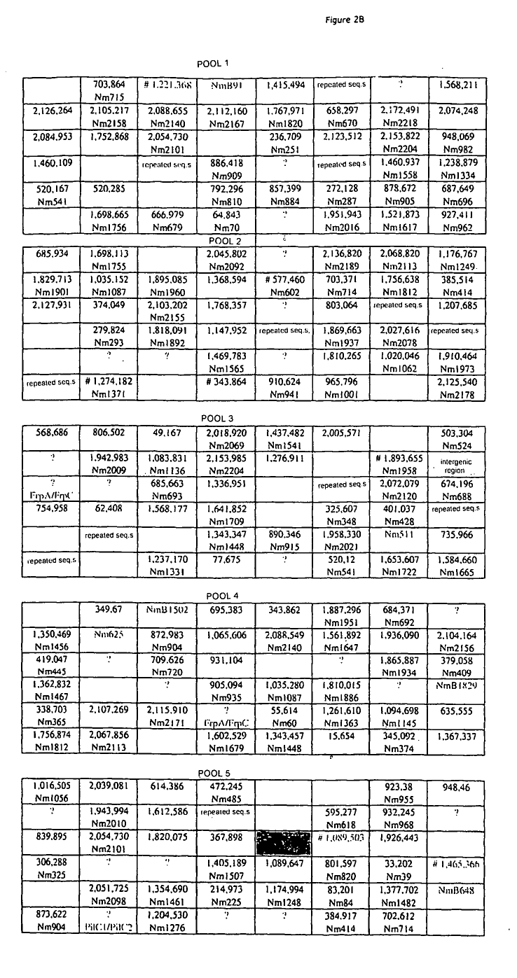

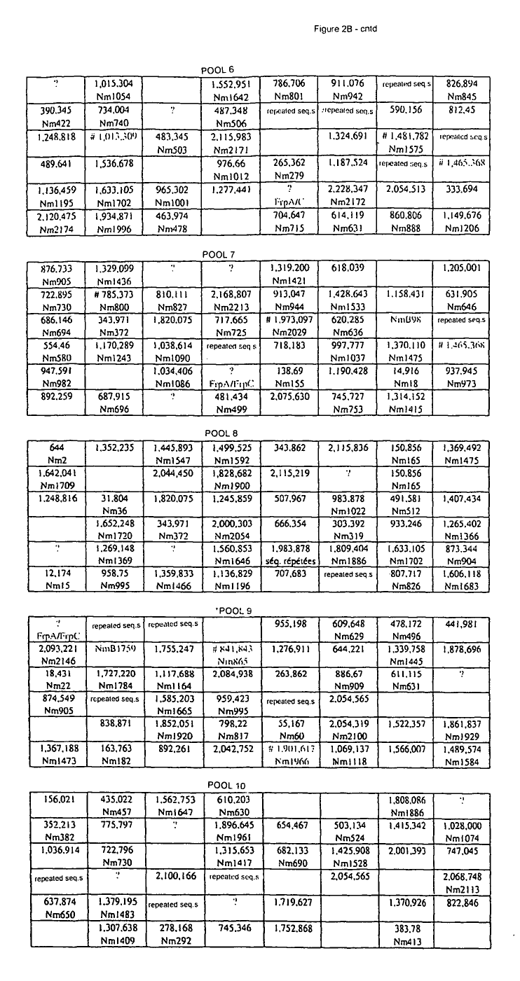

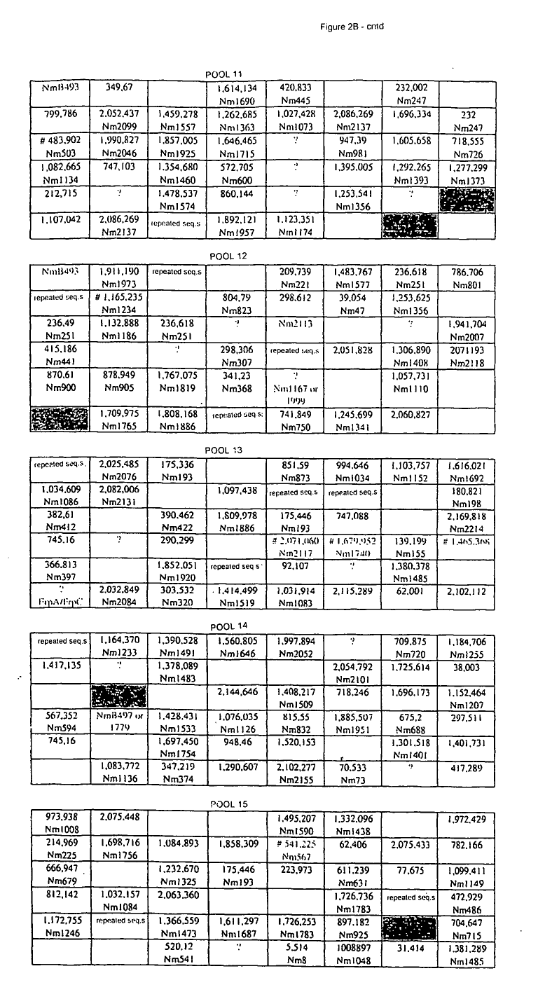

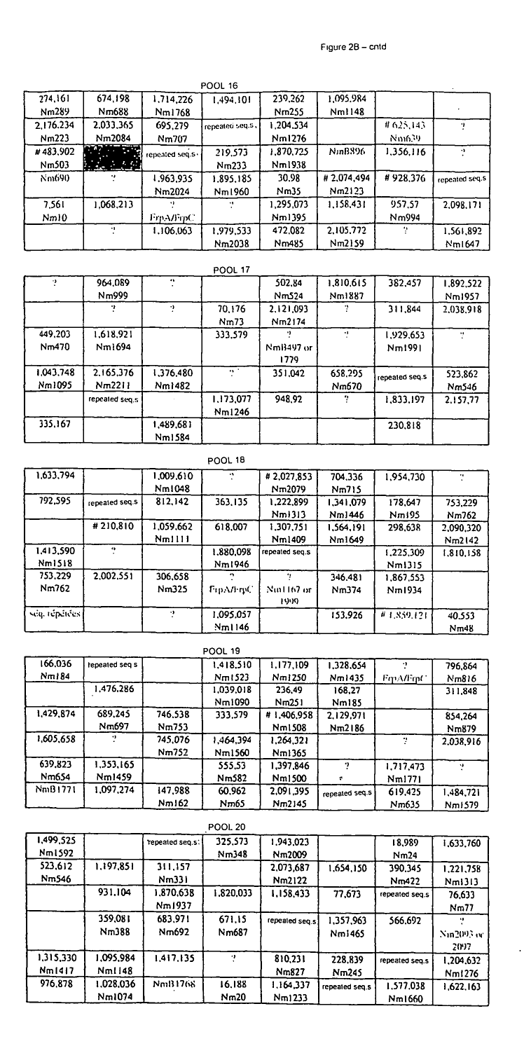

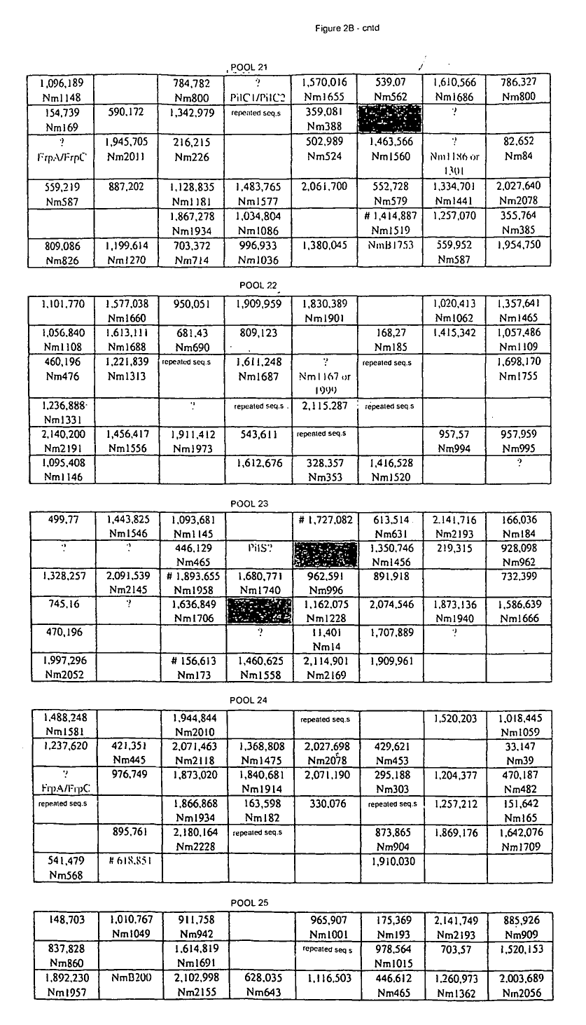

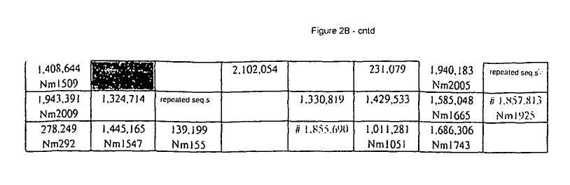

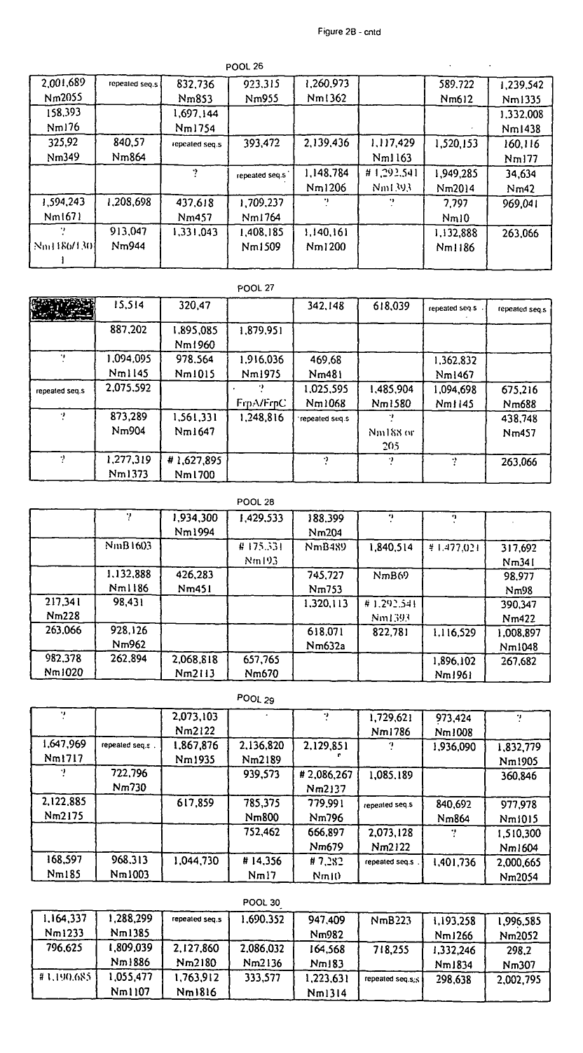

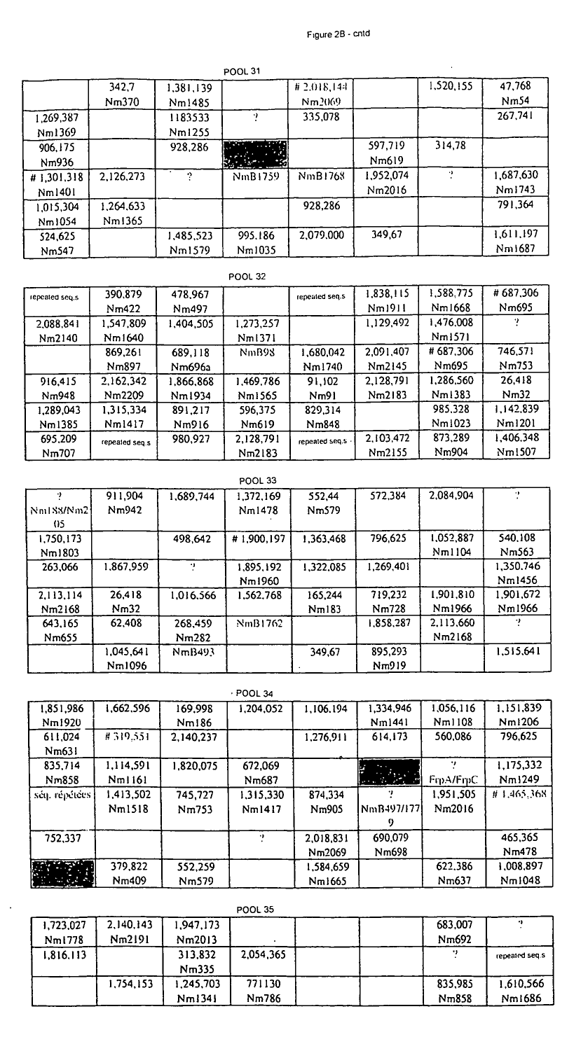

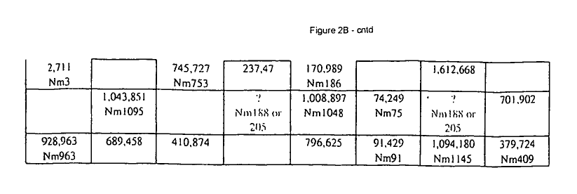

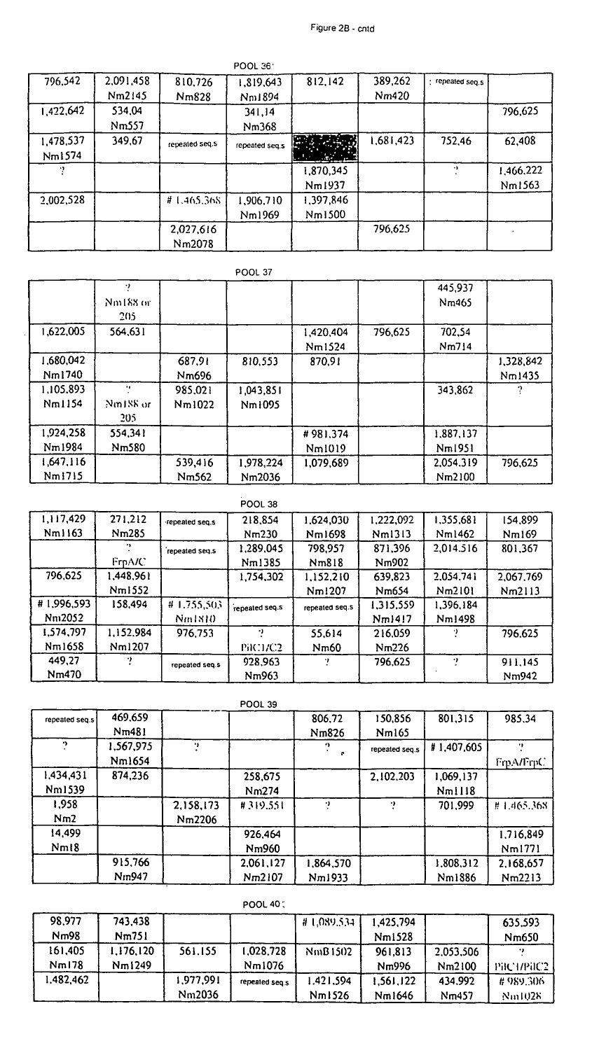

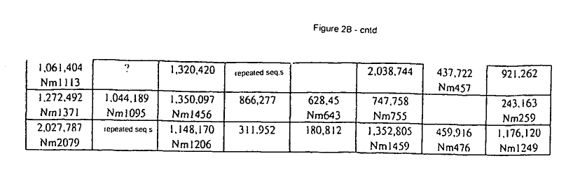

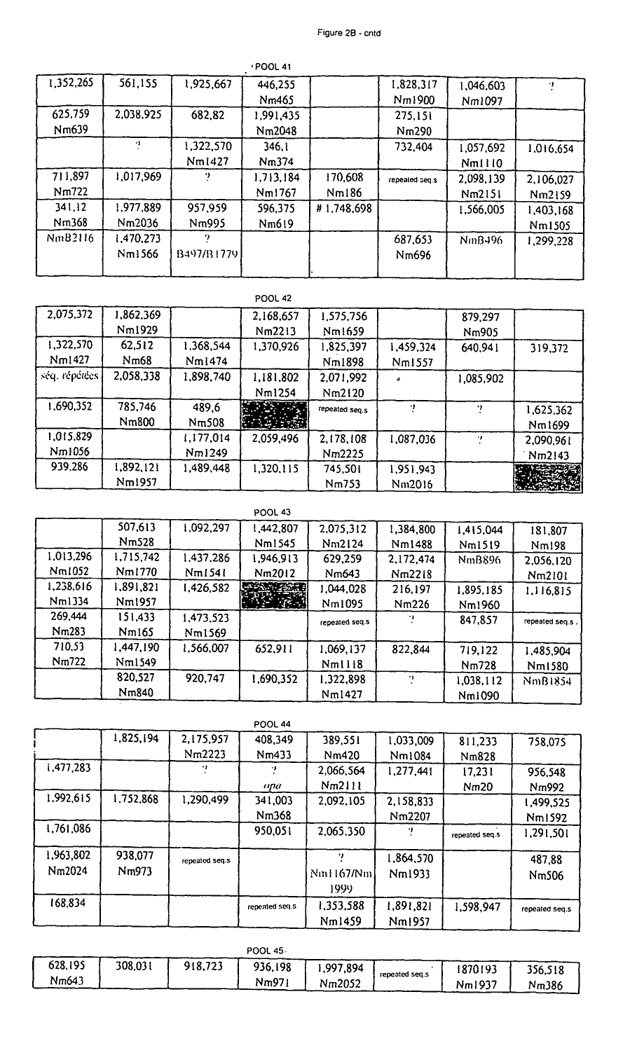

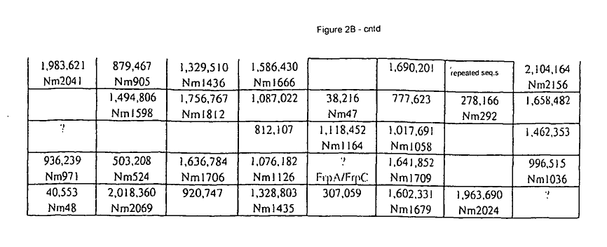

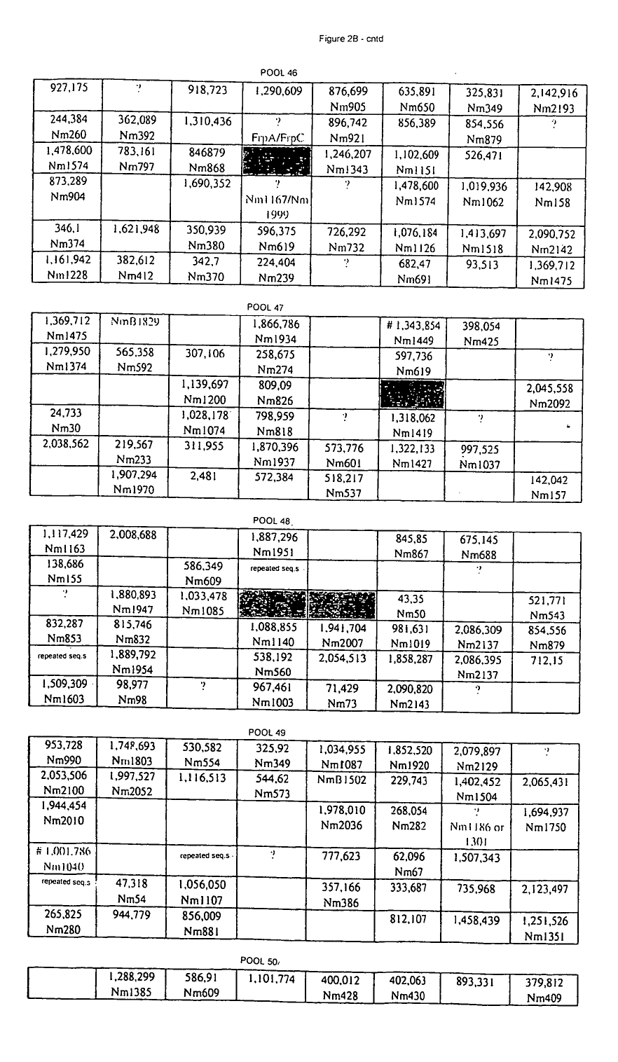

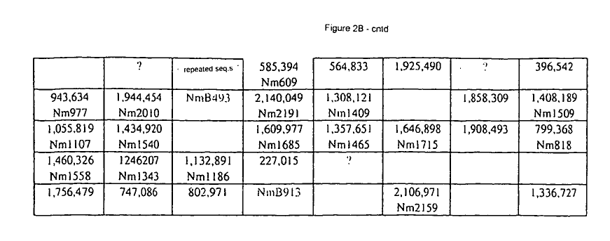

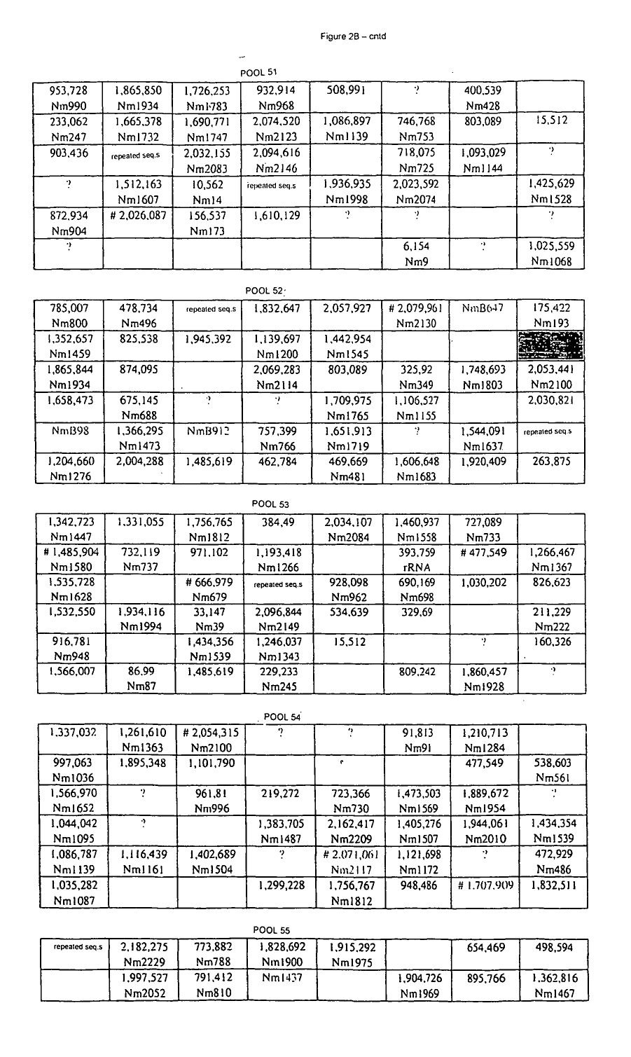

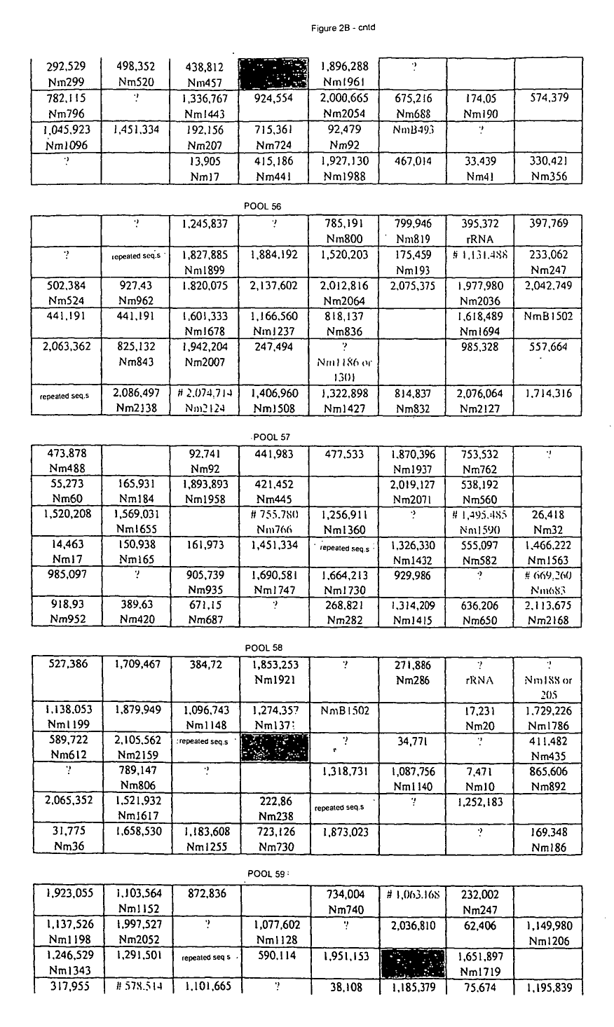

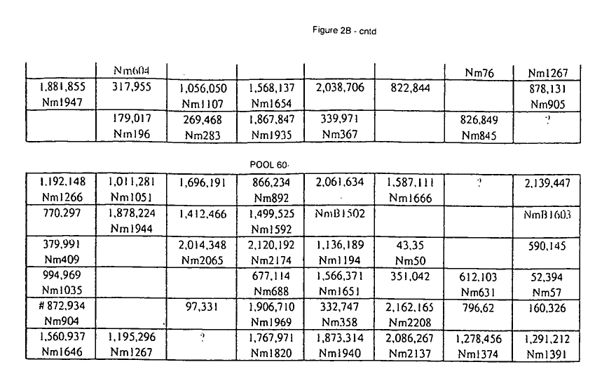

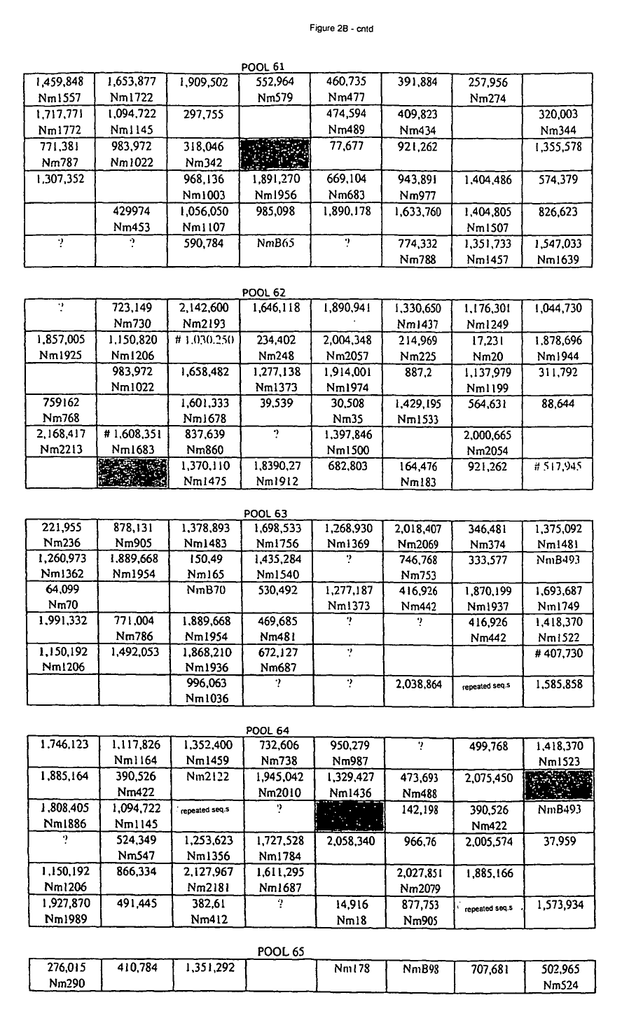

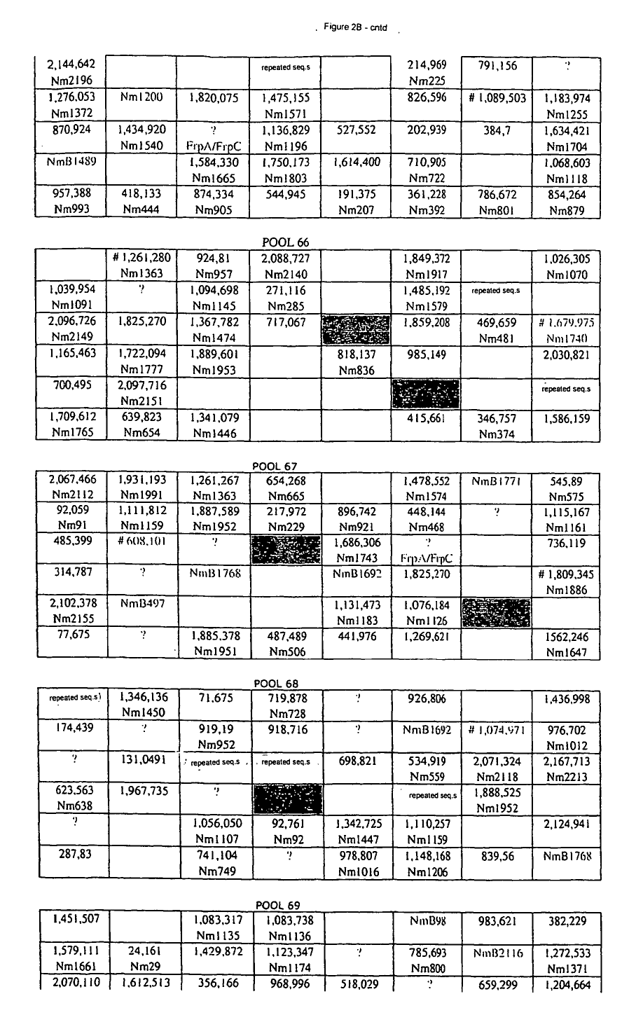

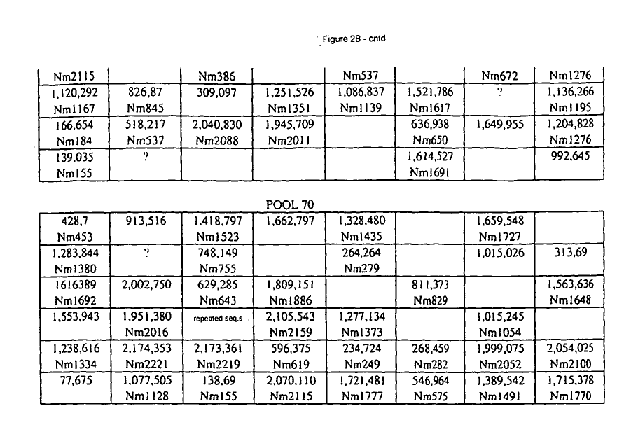

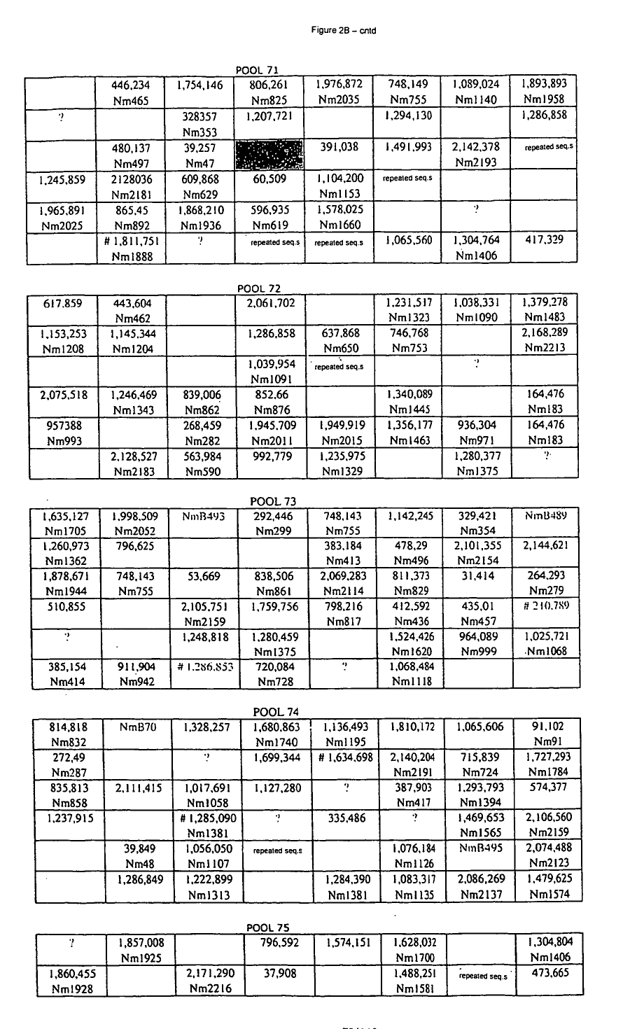

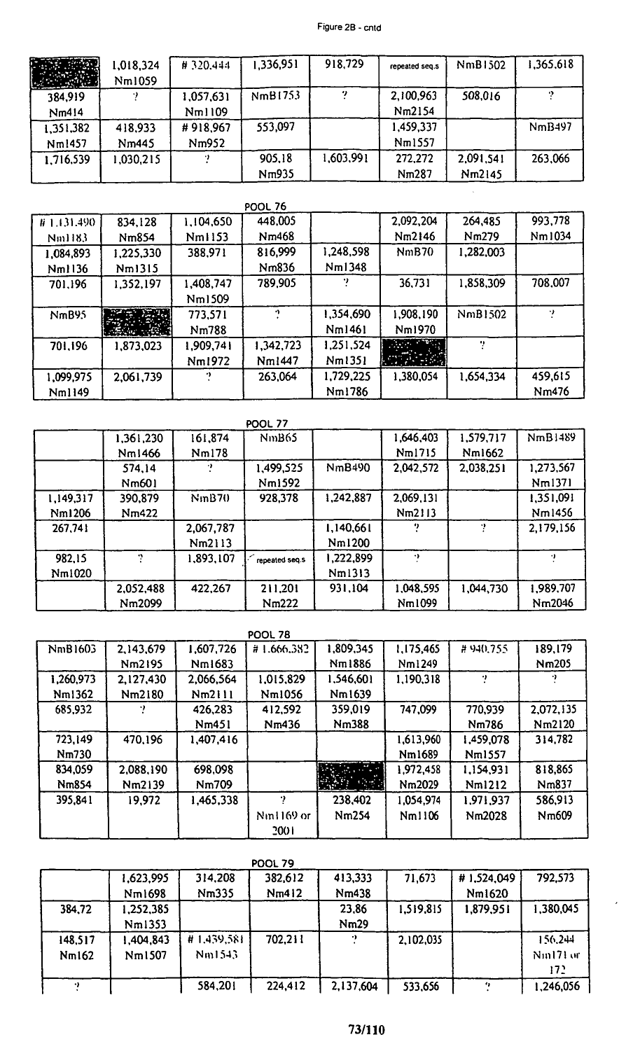

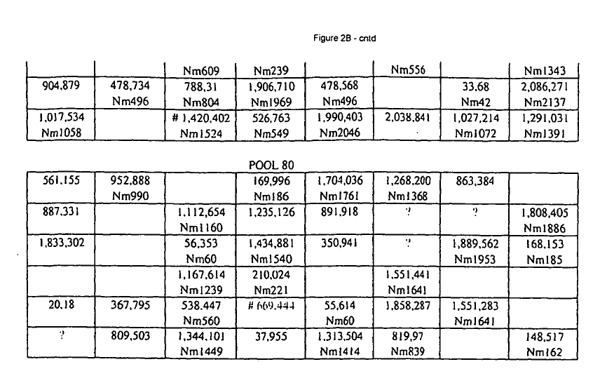

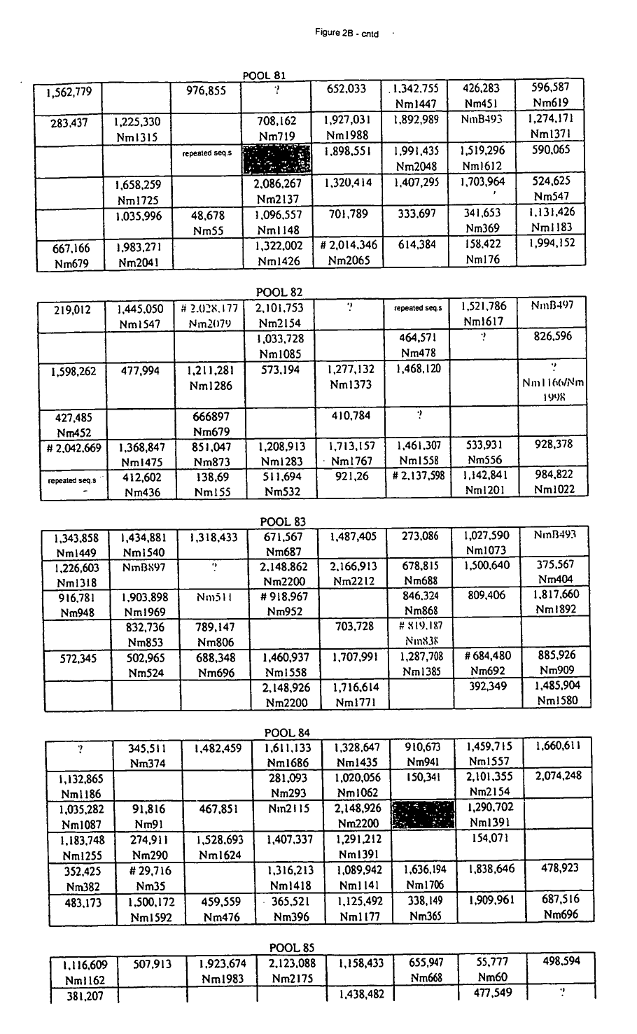

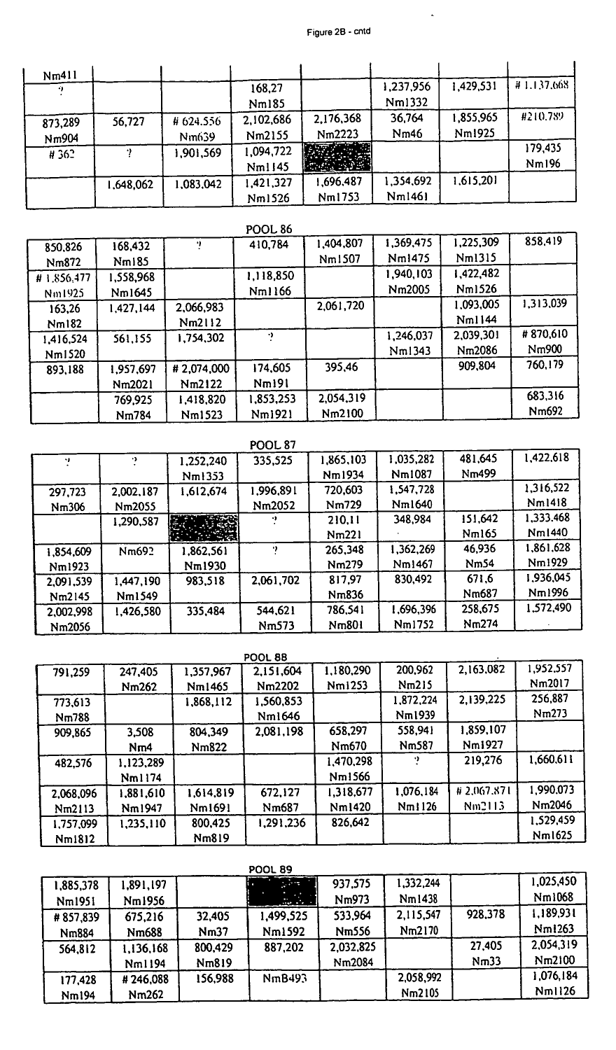

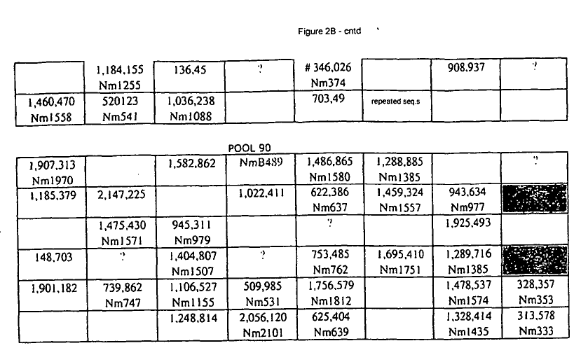

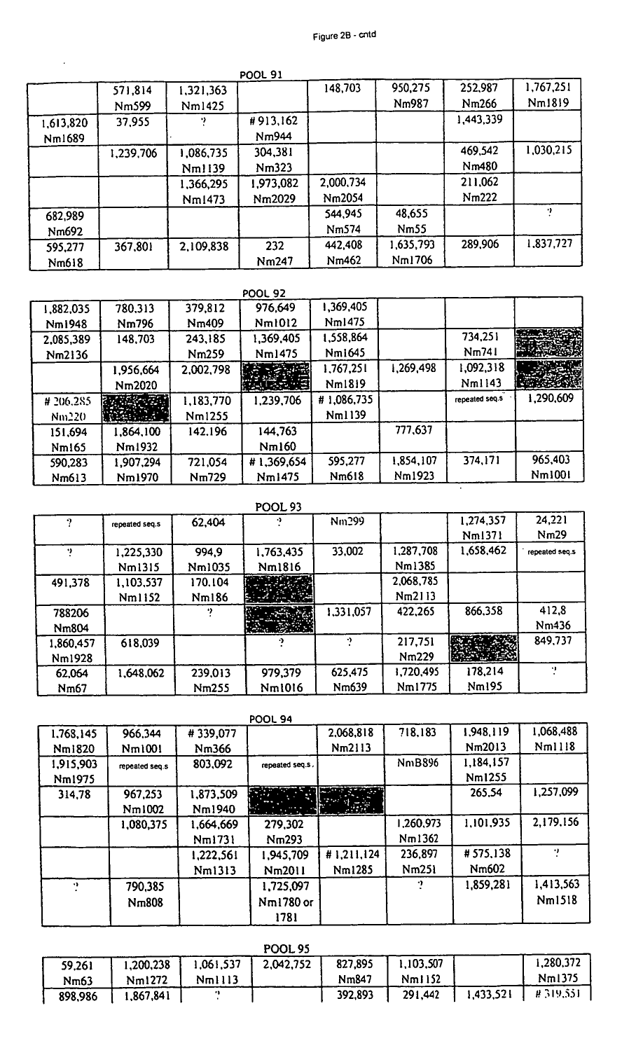

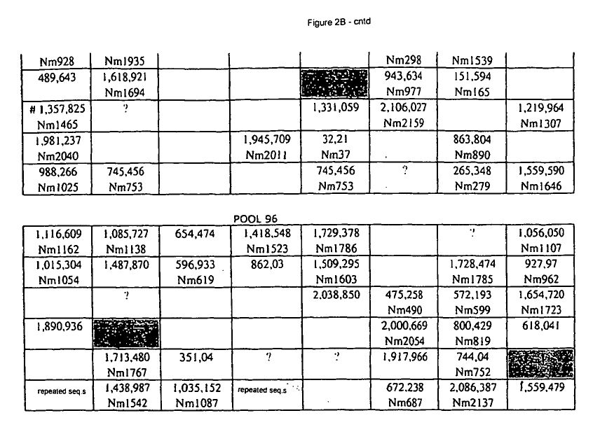

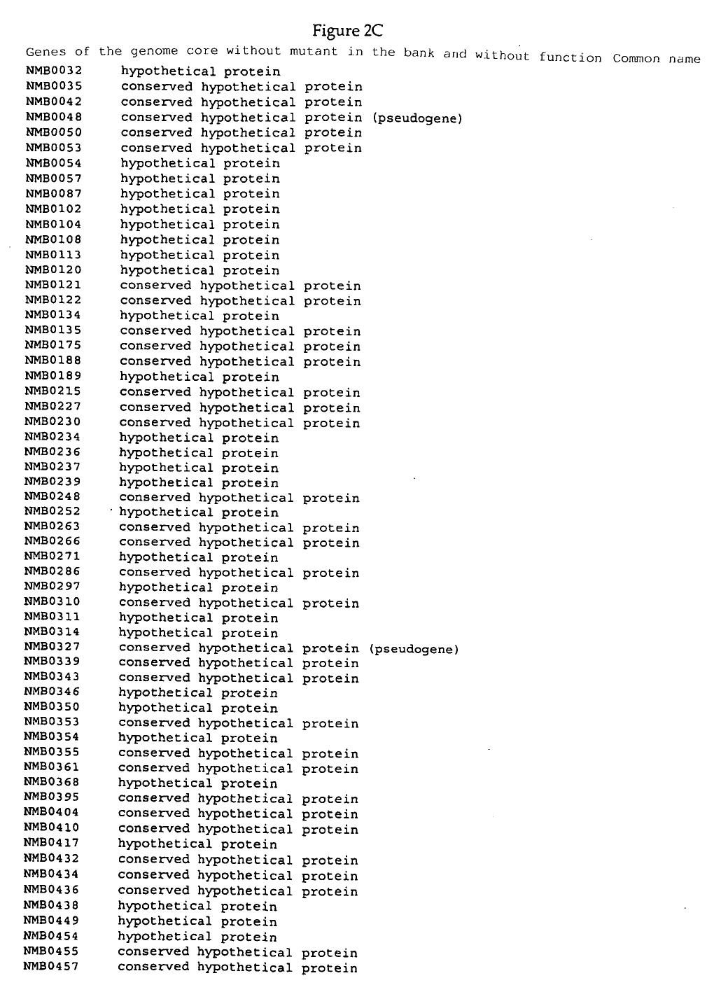

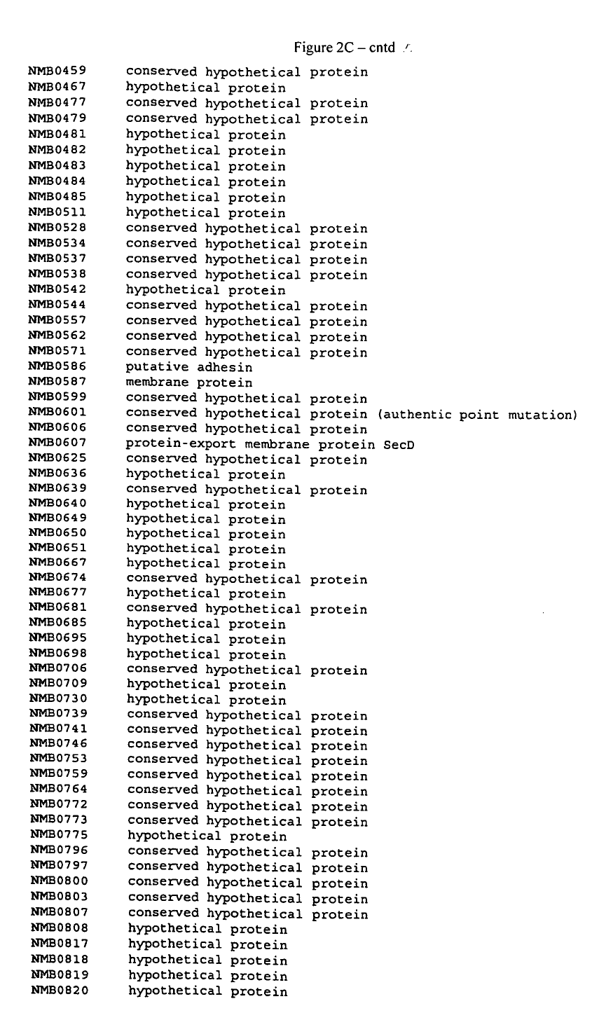

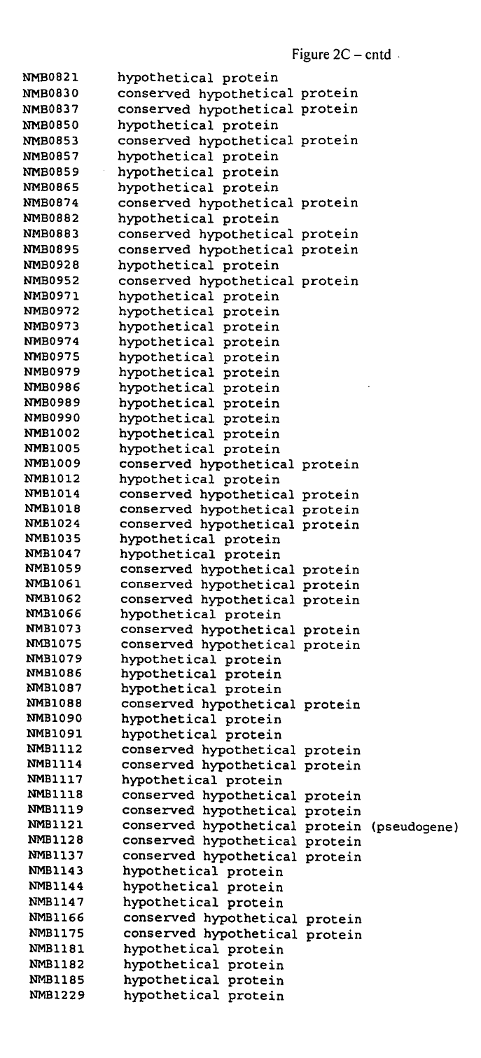

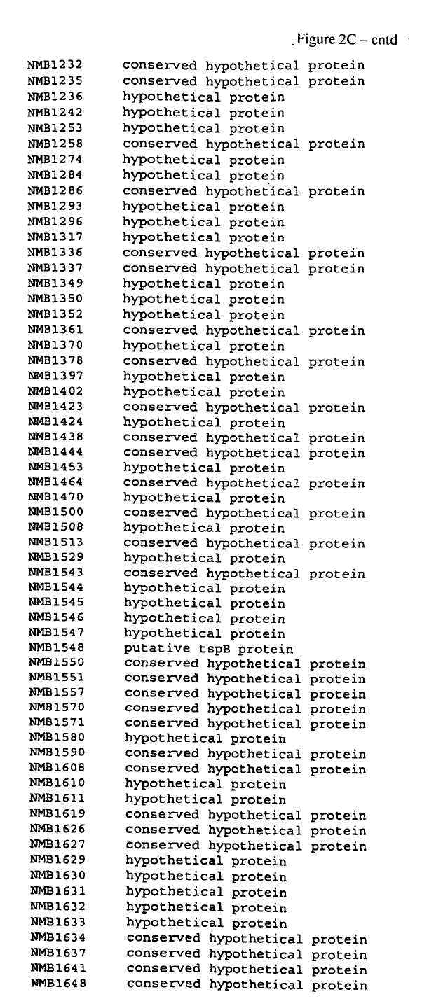

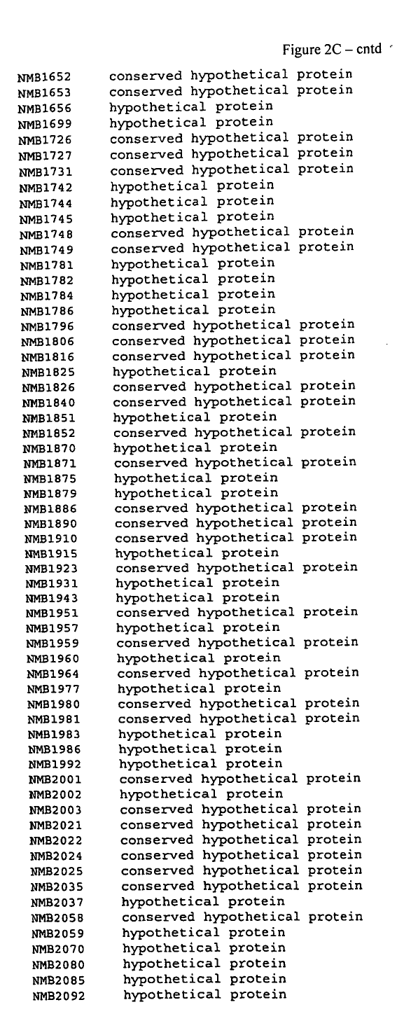

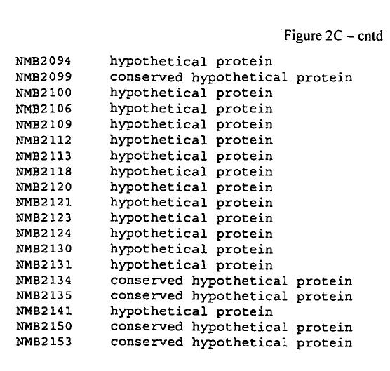

In particular the invention relates to the genes involved in the growth of bacteria in serum, chosen from the genes of FIG. 3, identified with respect to the number of the pool of mutants of FIG. 2.

Quite particularly the invention relates to the isolated genes NmB 352, NmB 065, NmB 2076, NmB 638, NmB 828, NmB 825 and NmB 790as new products.

The invention also relates to the application of the genes selected in relation to the growth phenotype in serum, as anti-pathogenicity targets, which consists of inhibiting the growth of Nm in vivo in serum.

Therefore the invention also relates to the application of these genes for screening and manufacturing medicaments allowing the opening of the blood-brain barrier to therapeutic ingredients, such as medicaments for Parkinson's Disease, Alzheimer's disease, antimitotics, medicaments for multiple sclerosis, antivirals, antimycotics and antibiotics and to allow prophylaxis for Nm infections with the development of vaccines.

Moreover the invention relates to the essential genes of Nm for which no mutant is present in the bank and the application of these genes as targets for developing antibiotics.

Other genes of great interest according to the invention are characterized in this that they are involved in the interaction with endothelial cells. In particular reference is made to the genes of Tables 1 and 2, especially to NmA 1110, NmA 1111, NmA 1892, NmA 1107, NmA 1108, NmA 1109, and NmA 1523.

The proteins corresponding to those coded by these genes can be used for the development of vaccines. To that end, the proteins are purified, injected according to standard techniques into animals, for example into a rabbit, in order to produce antibodies. The antibodies are recovered and purified. Their bactericidal activity is verified in the presence of complement.

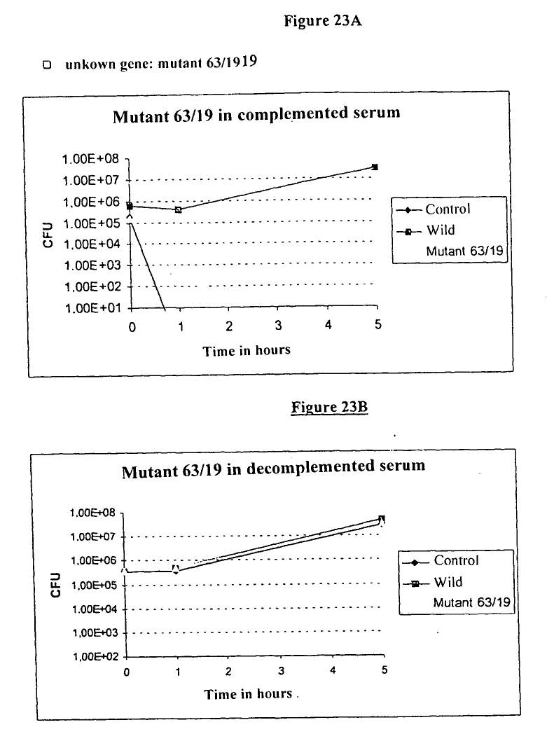

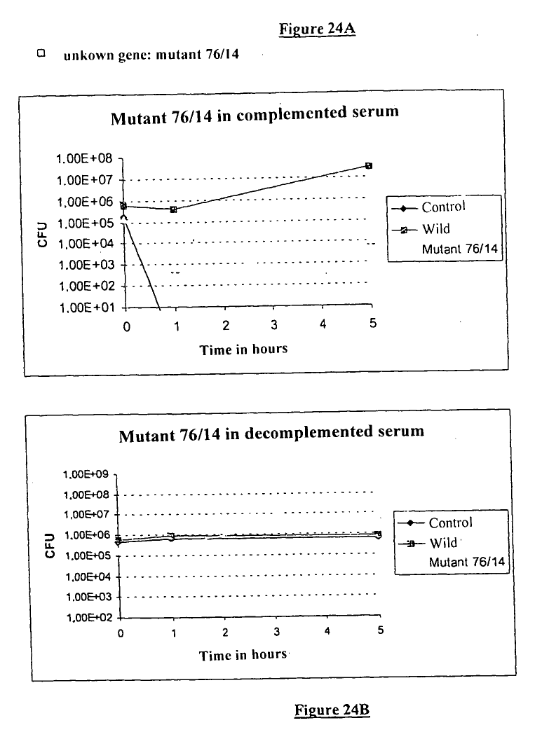

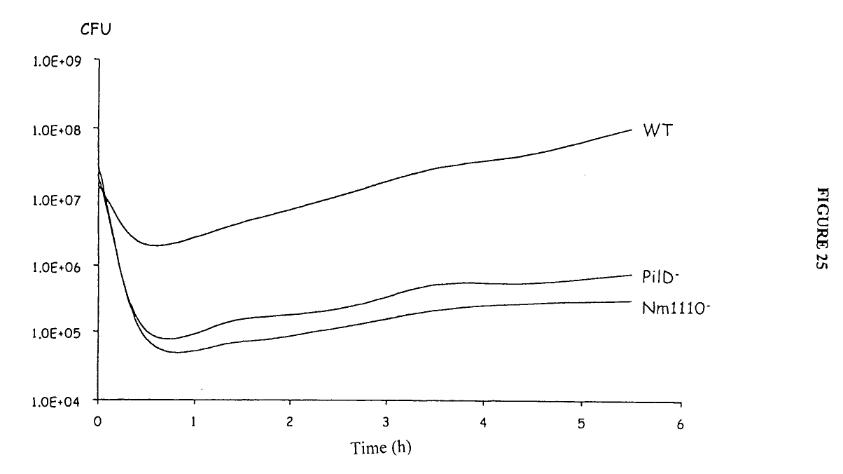

Given its extremely poor adhesion properties, as illustrated by FIG. 25, the protein Nm 1110 is particularly preferred for the development of vaccines.

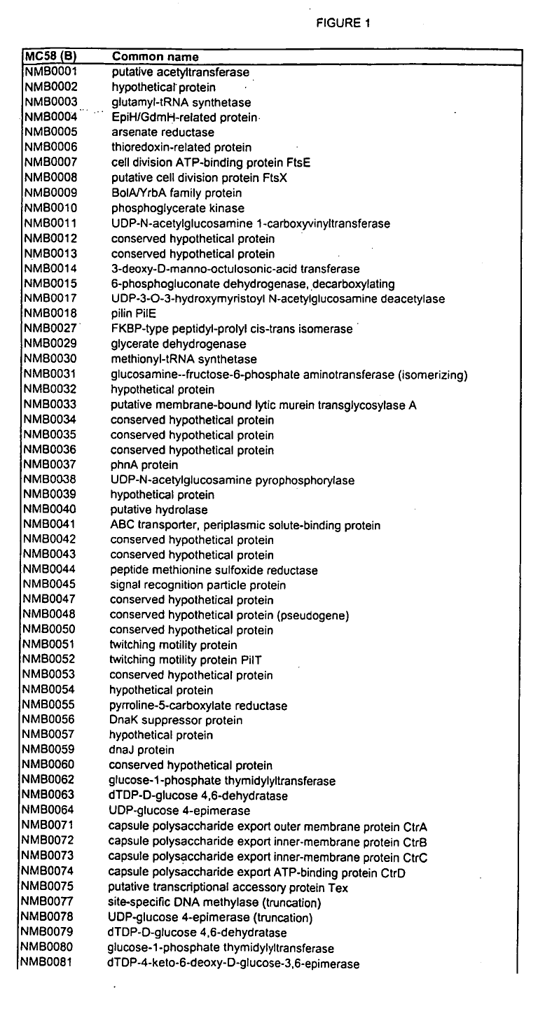

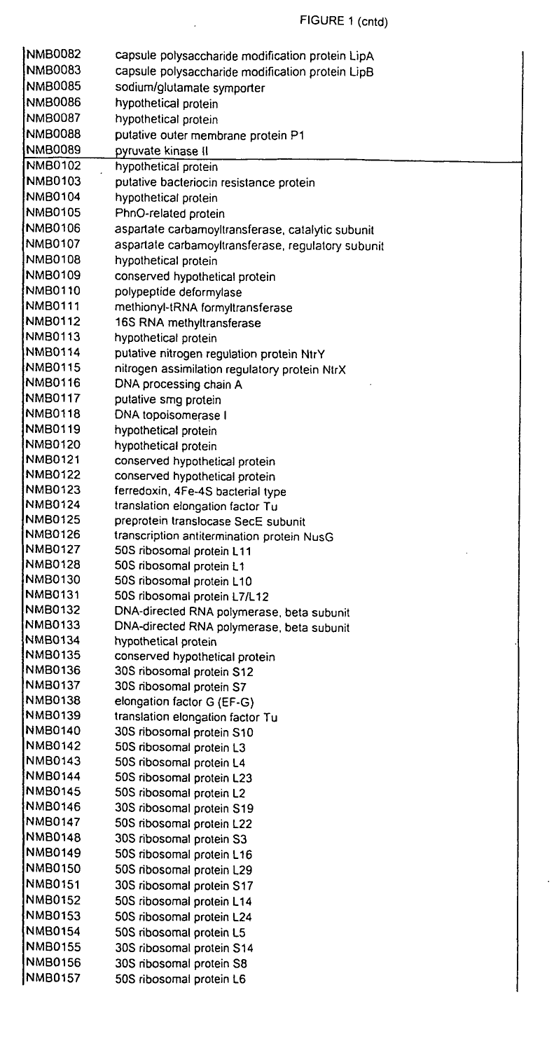

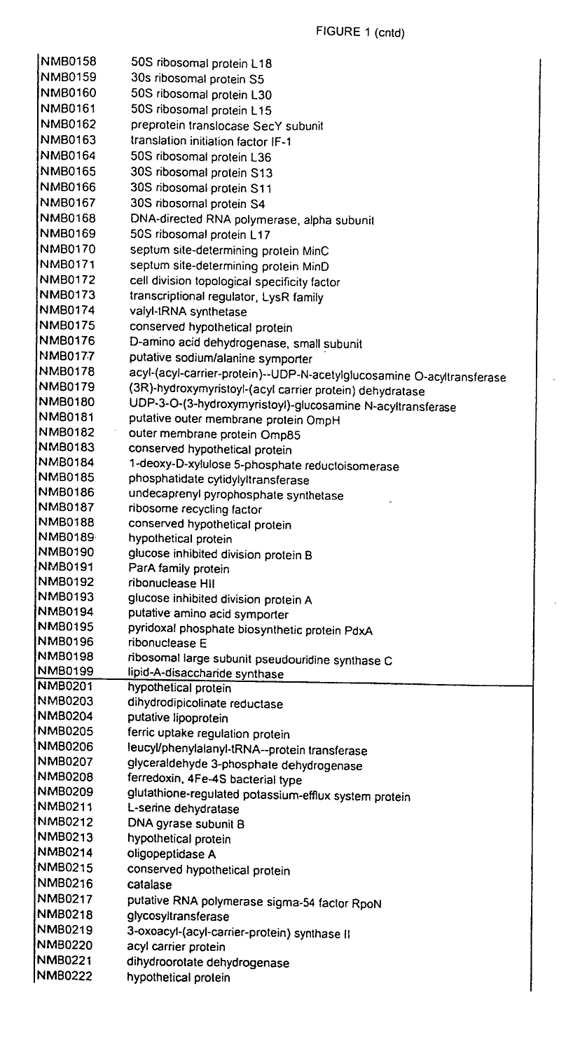

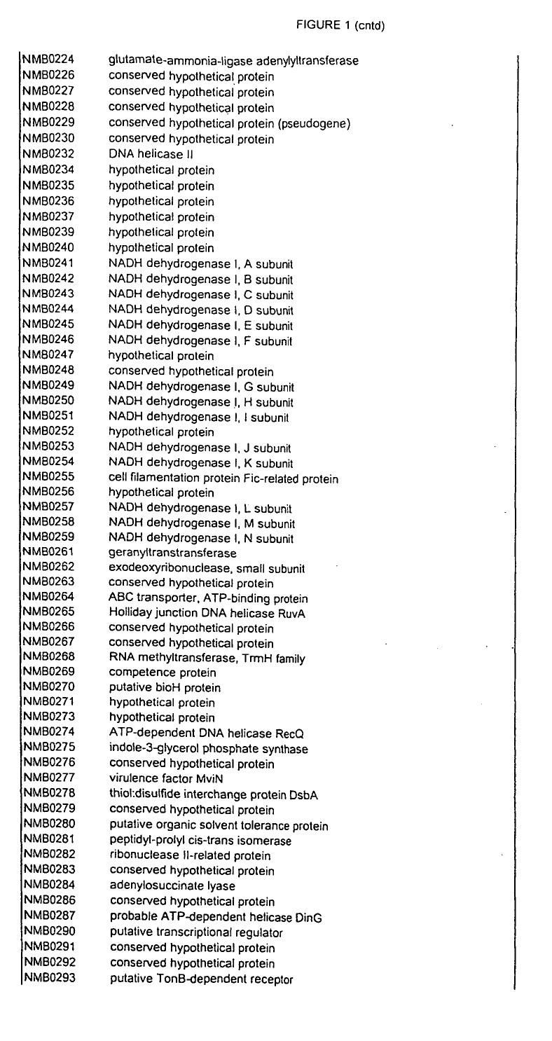

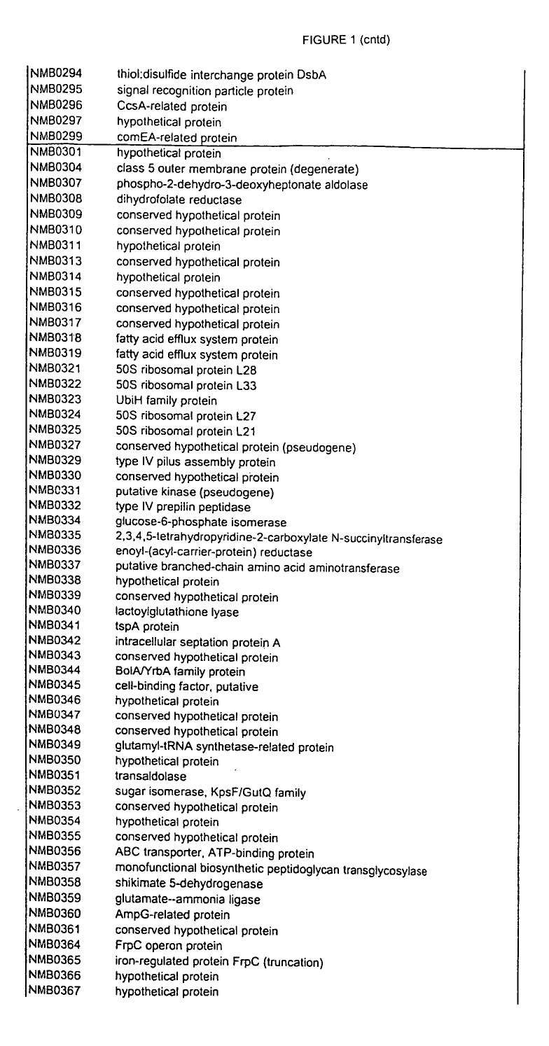

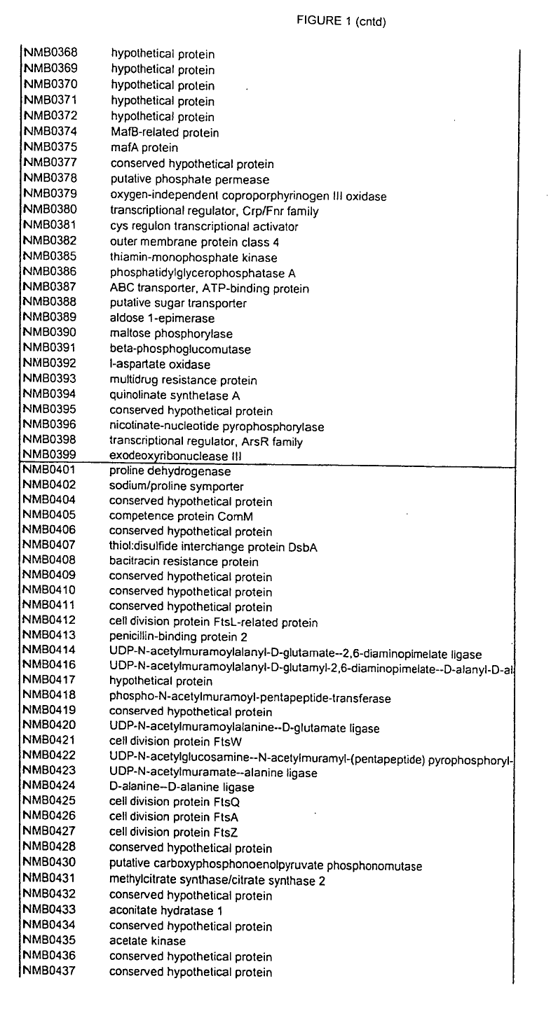

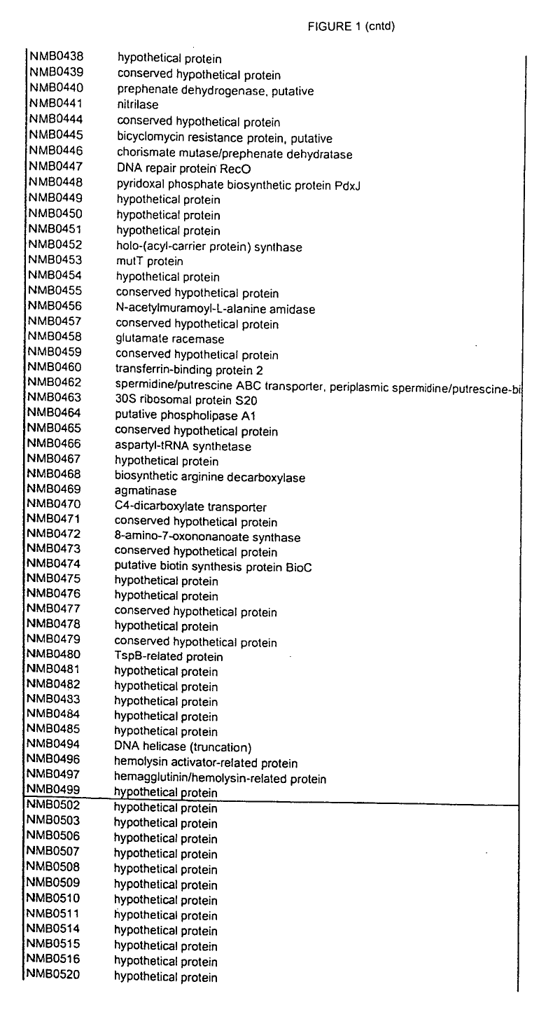

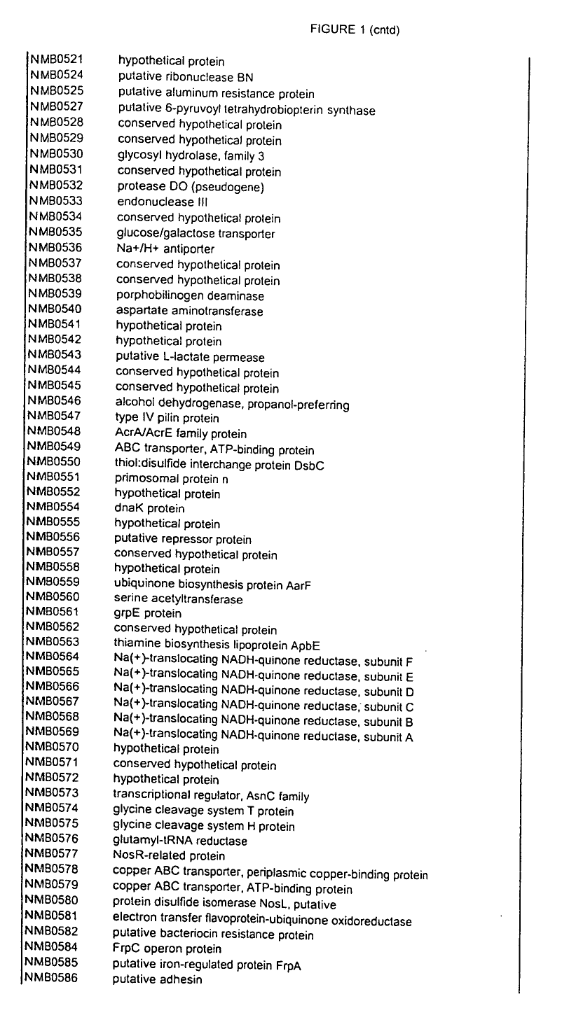

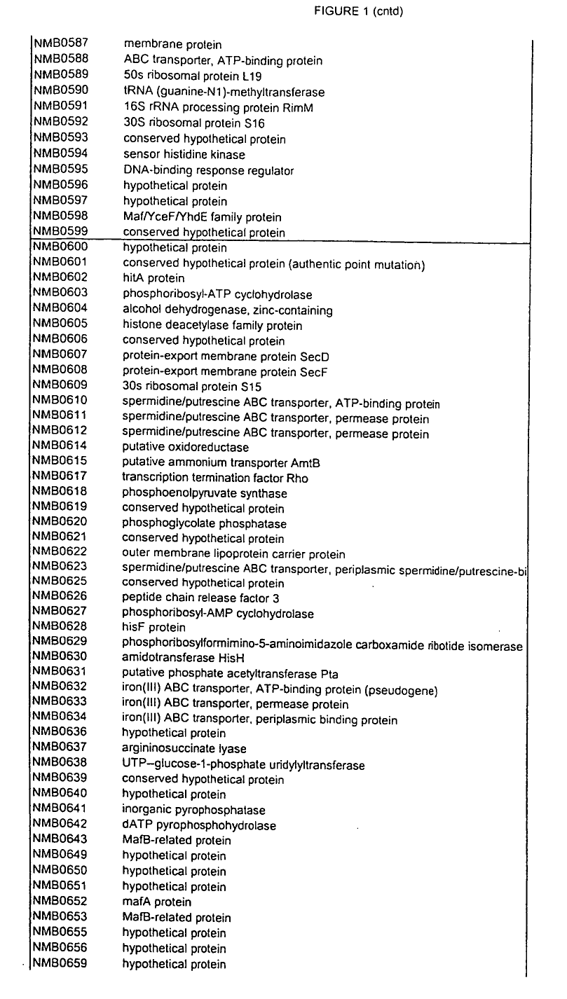

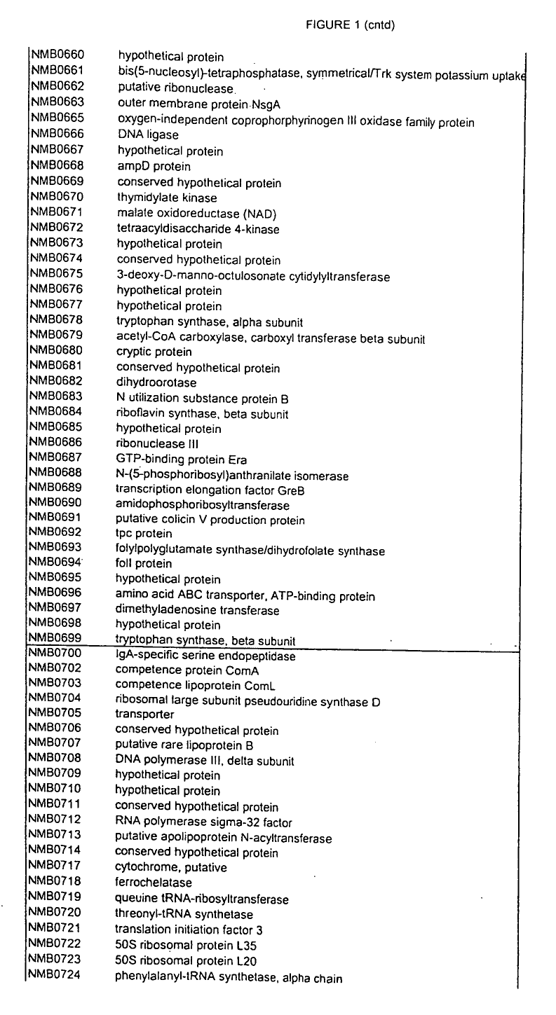

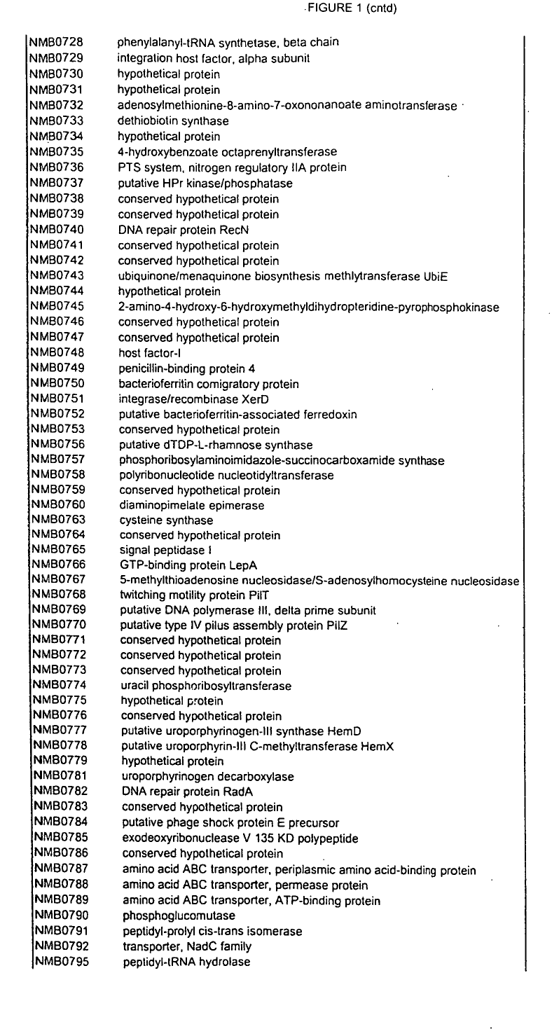

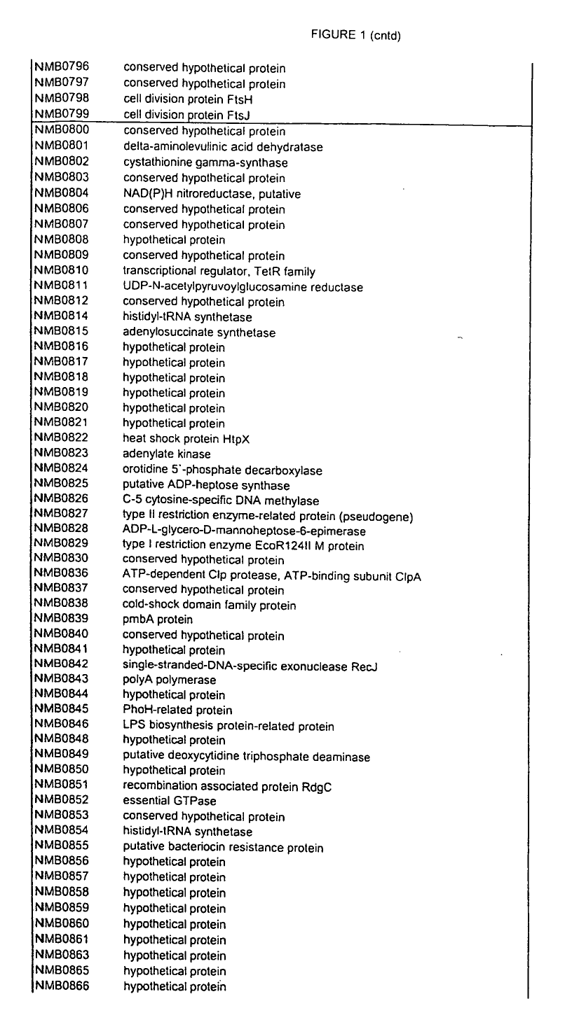

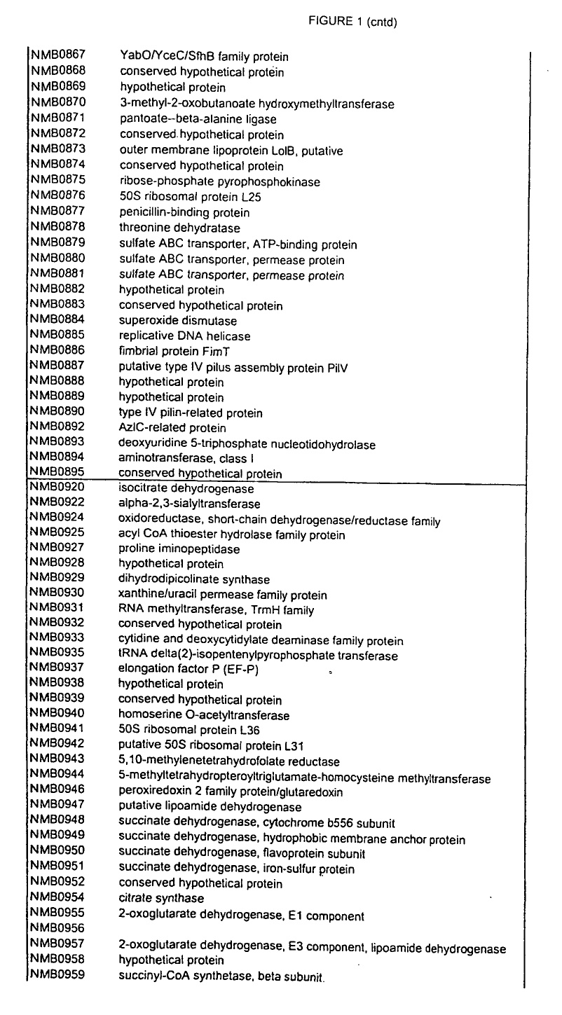

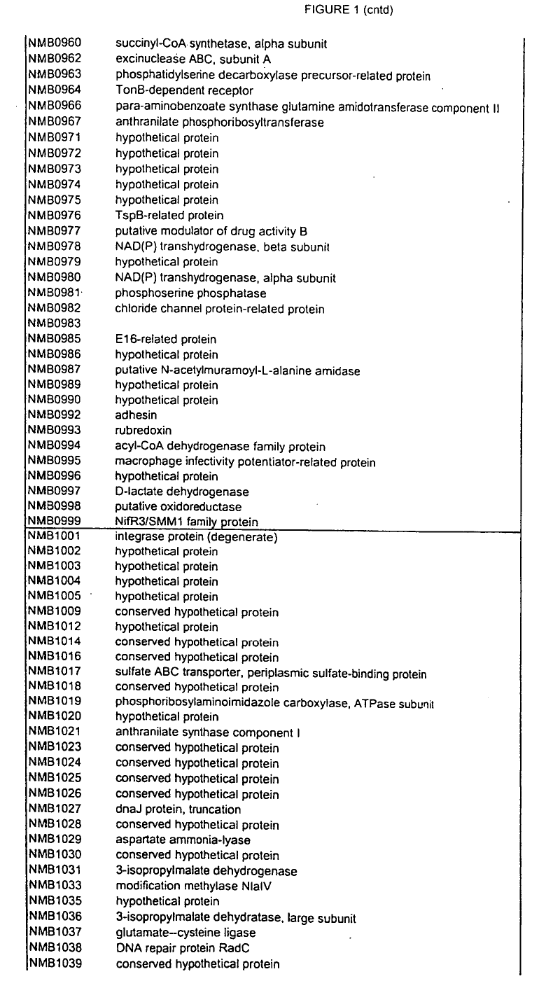

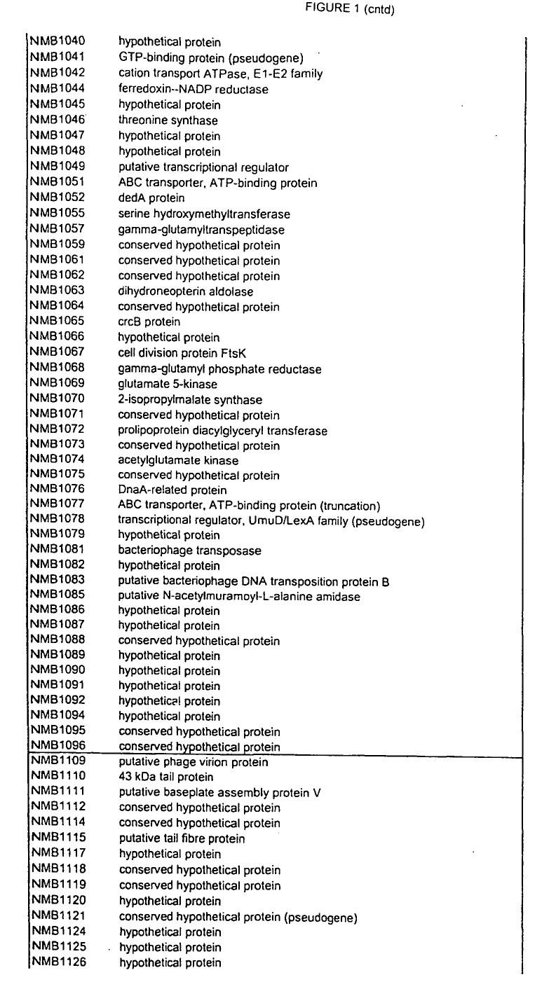

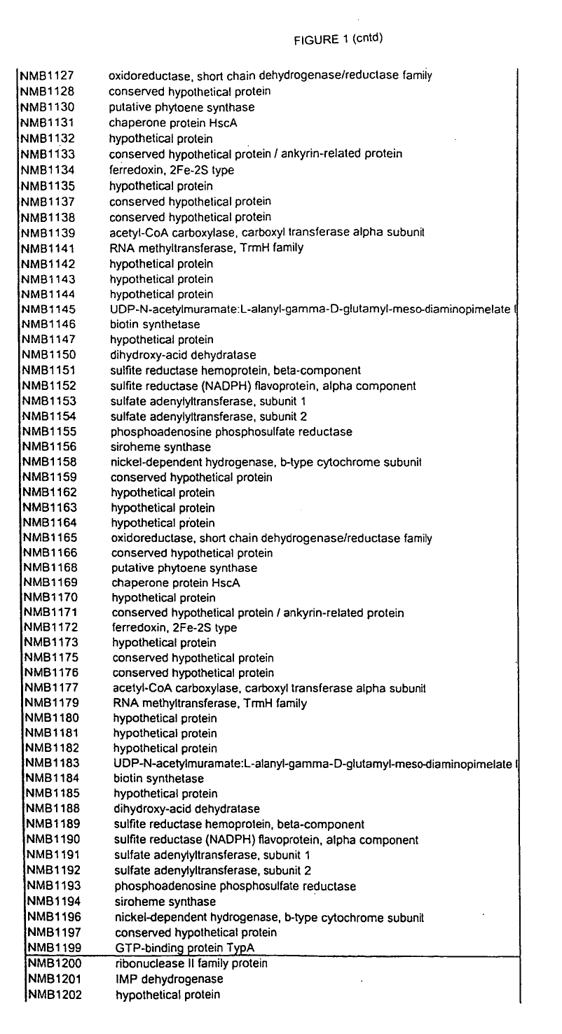

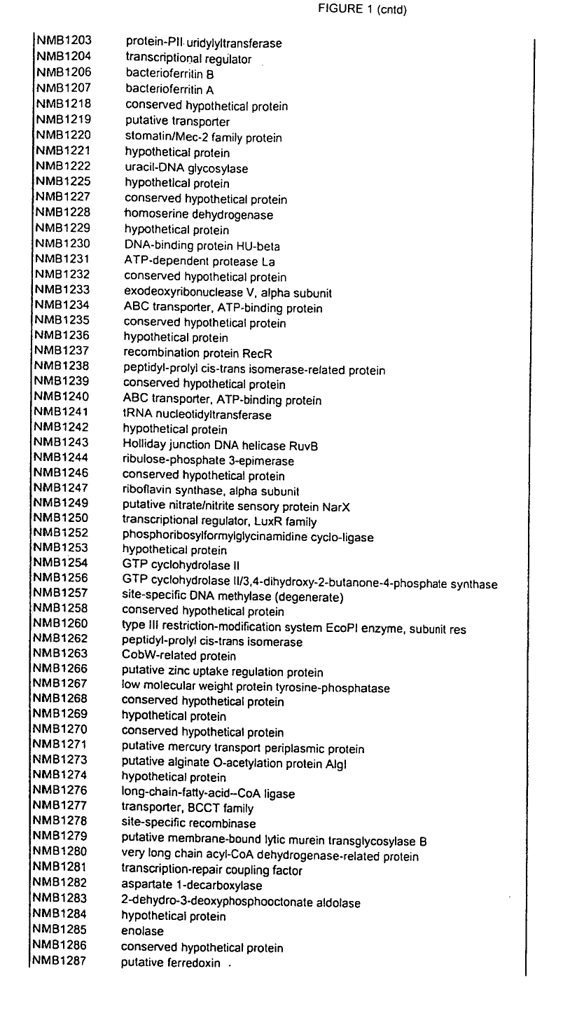

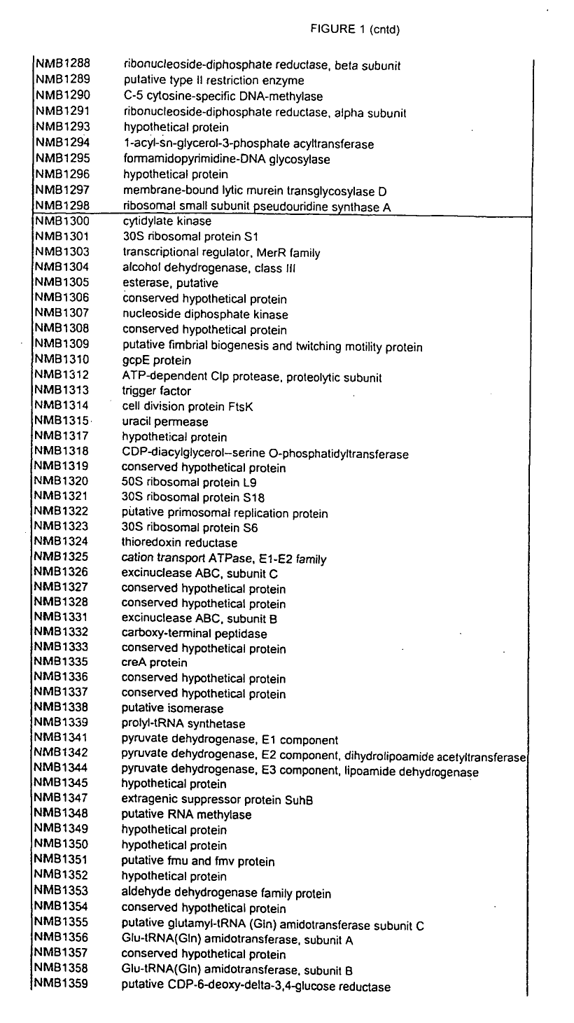

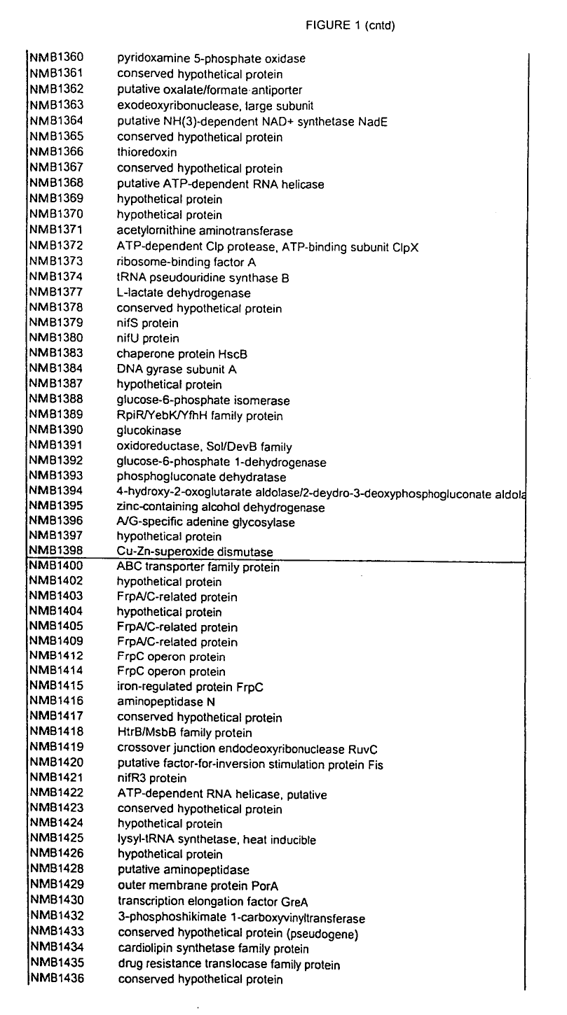

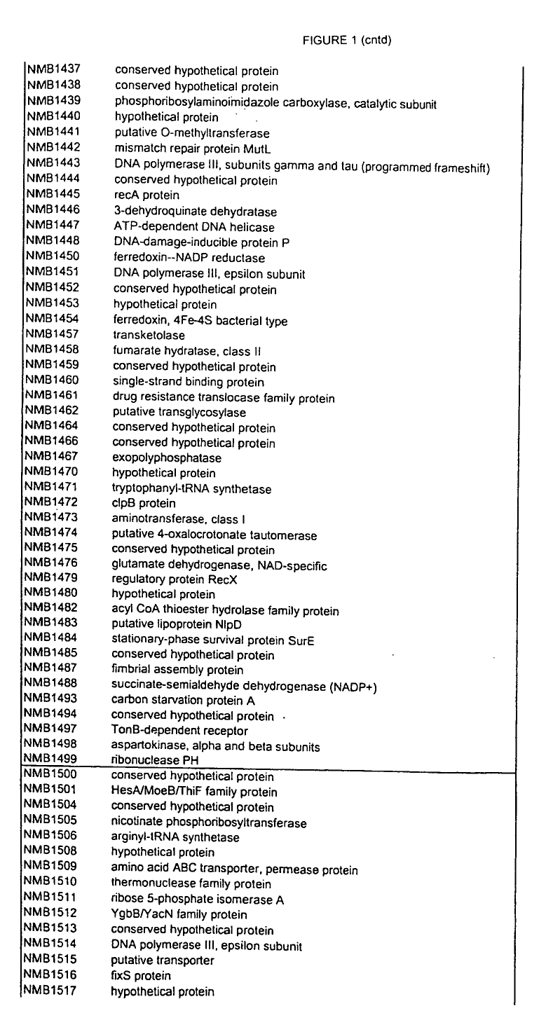

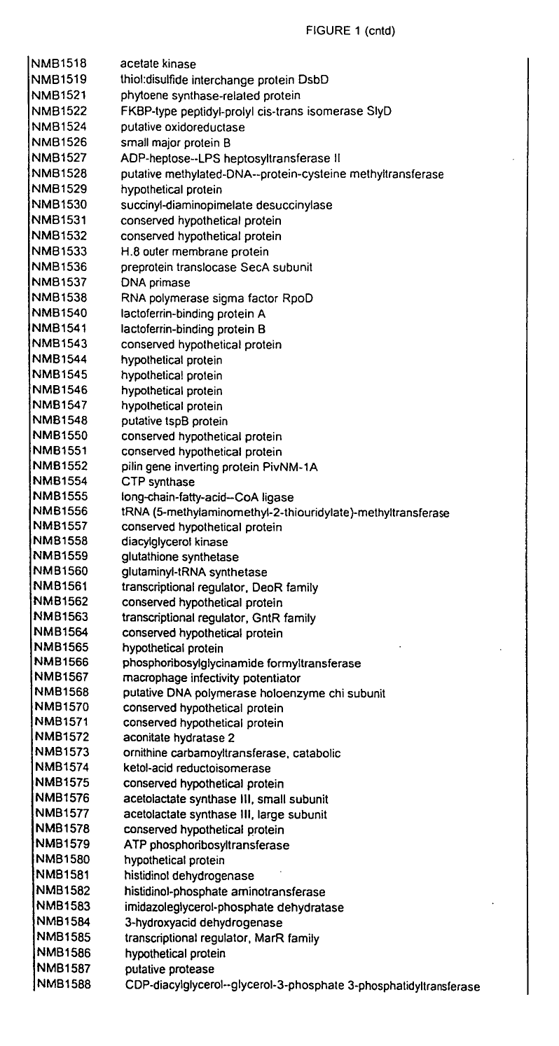

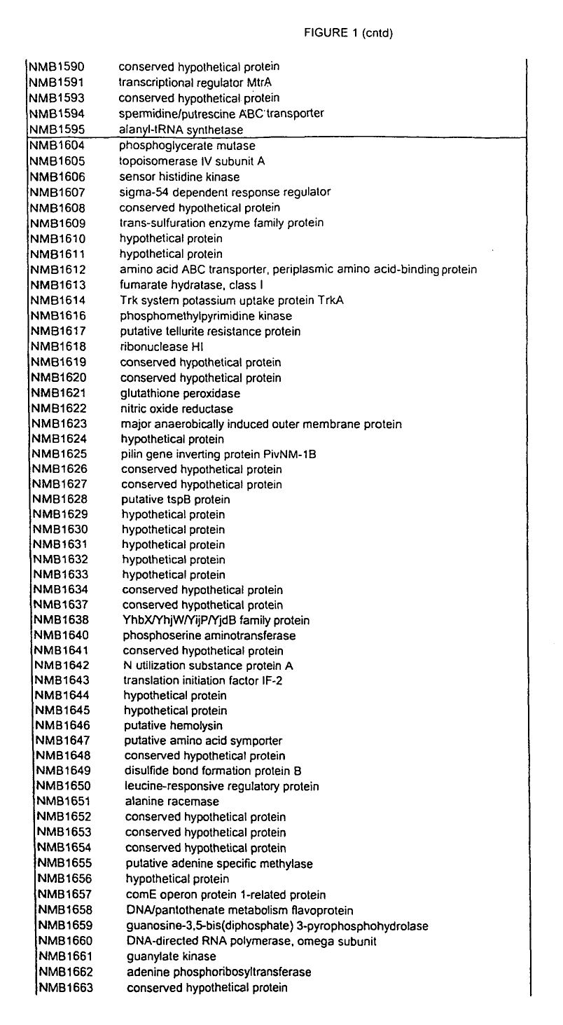

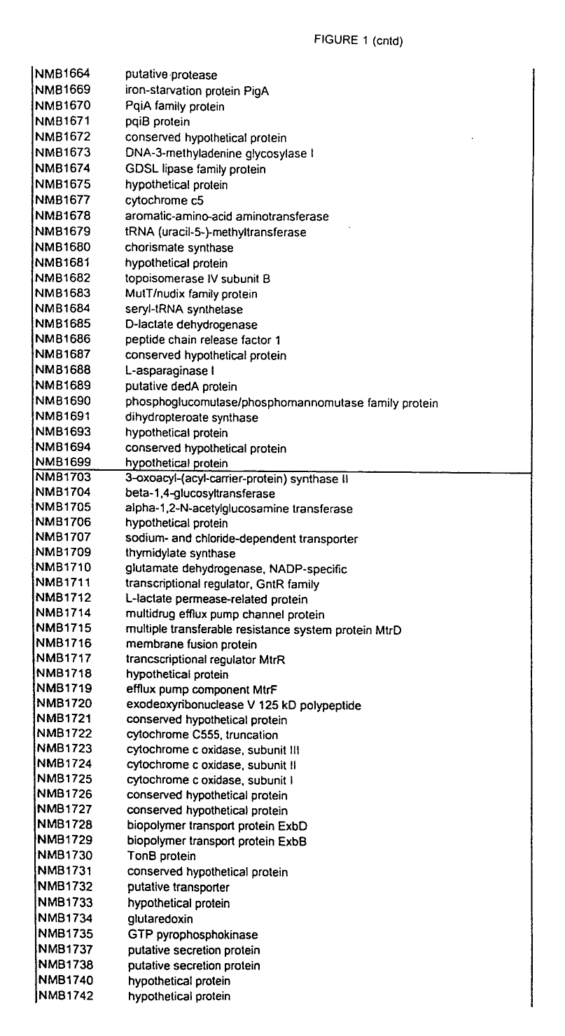

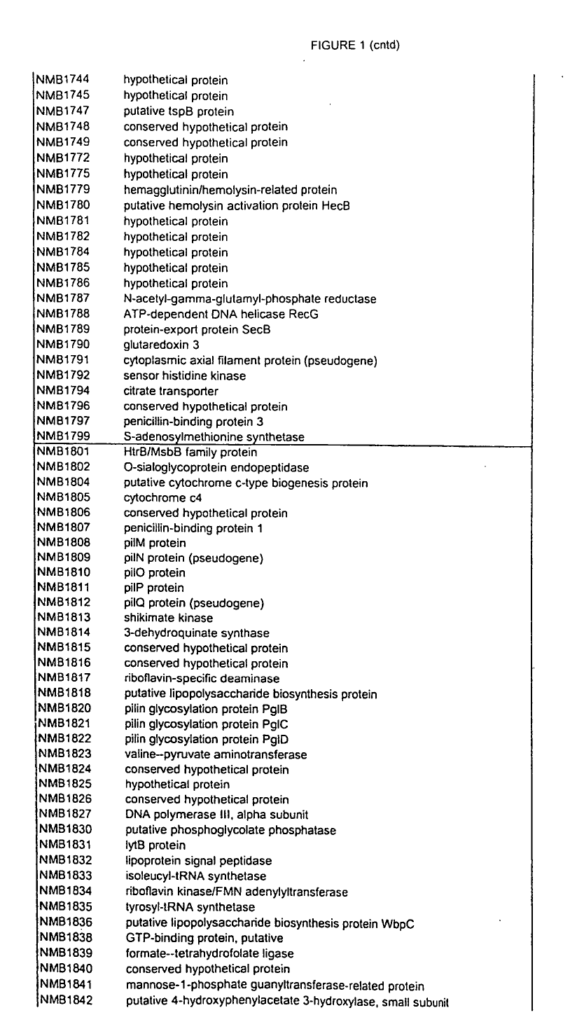

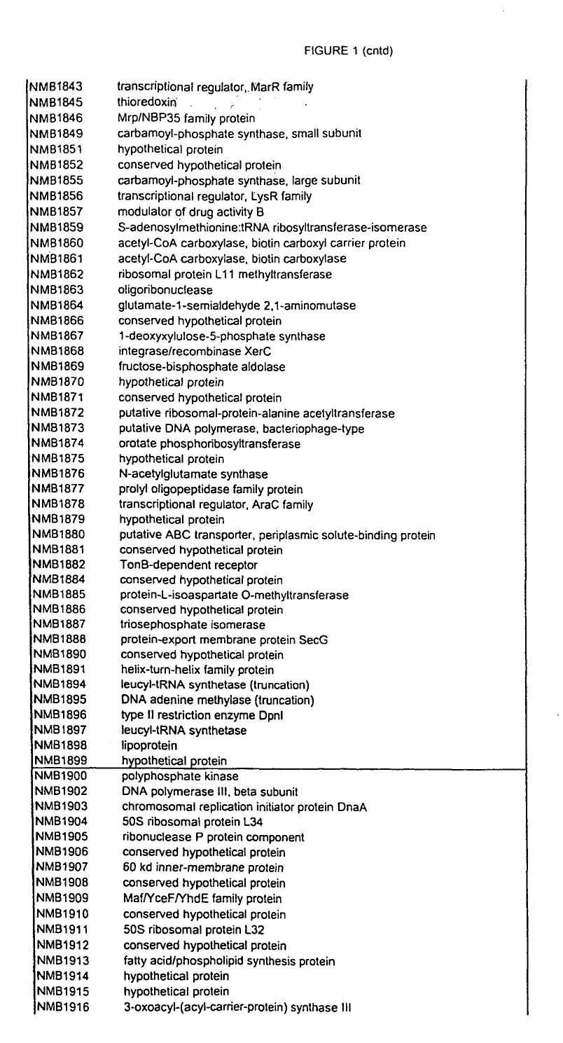

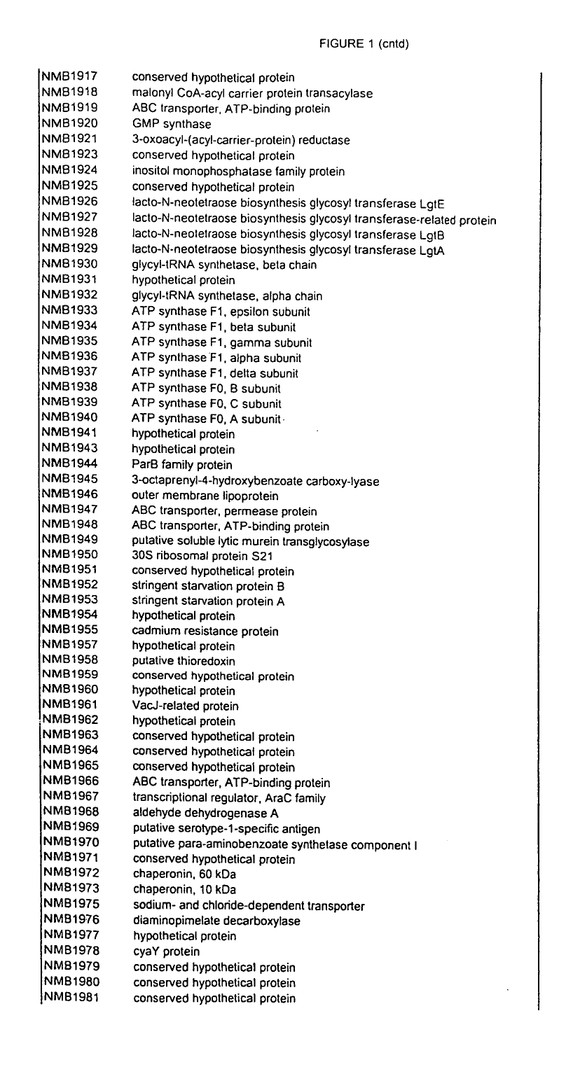

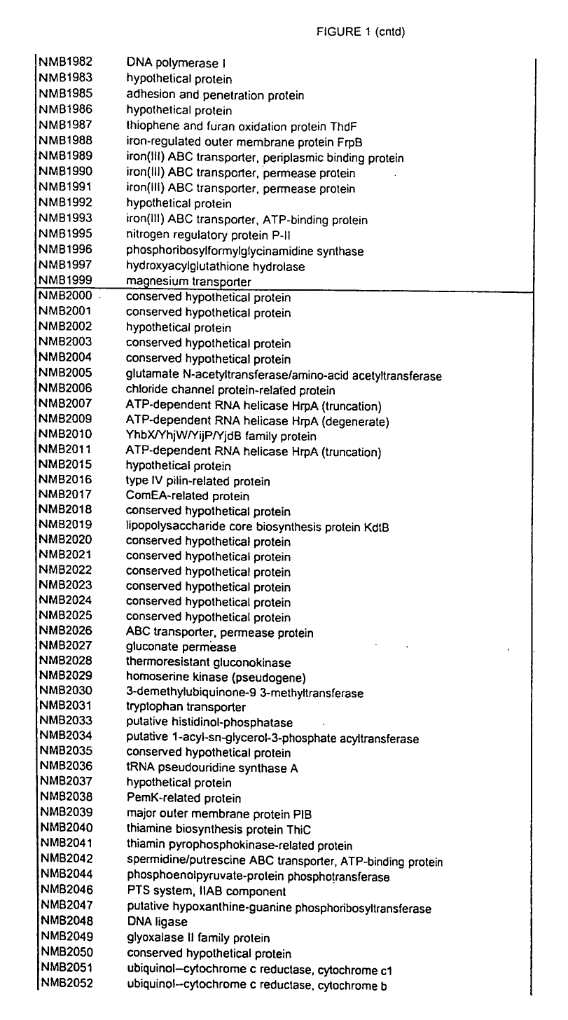

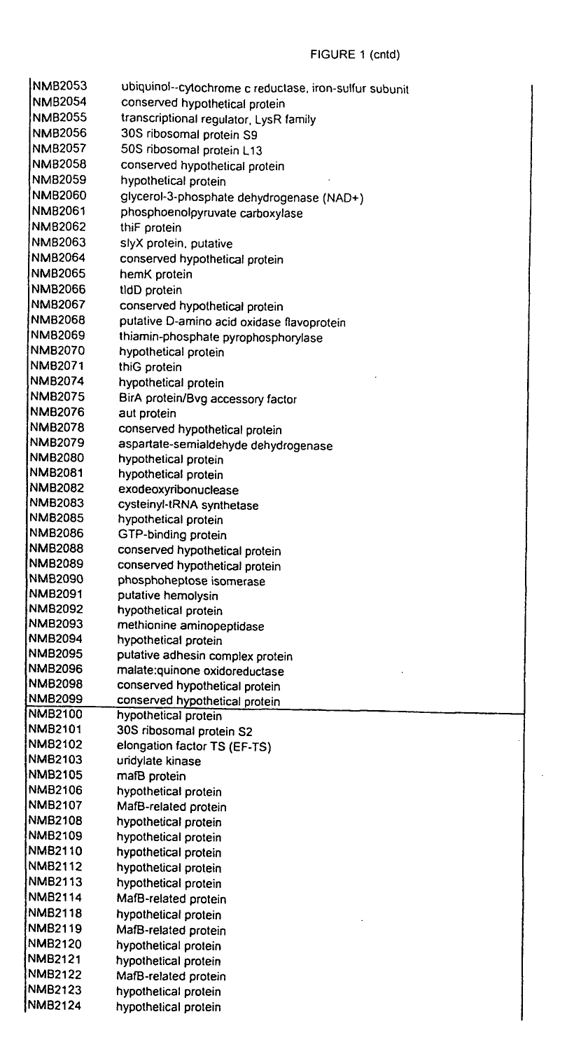

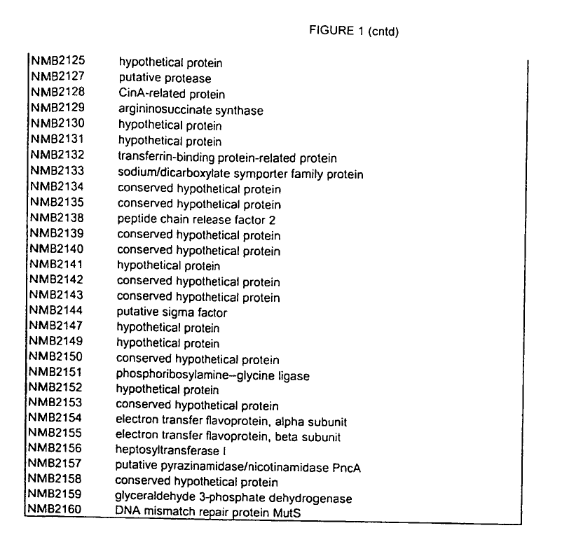

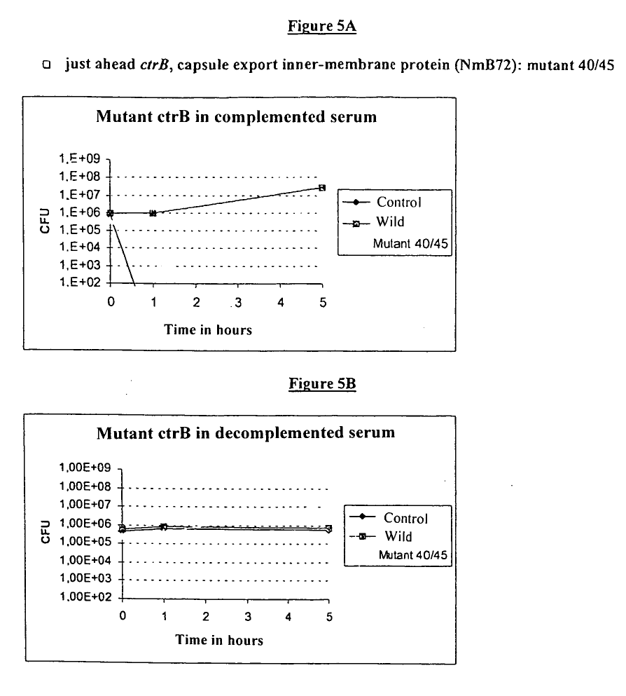

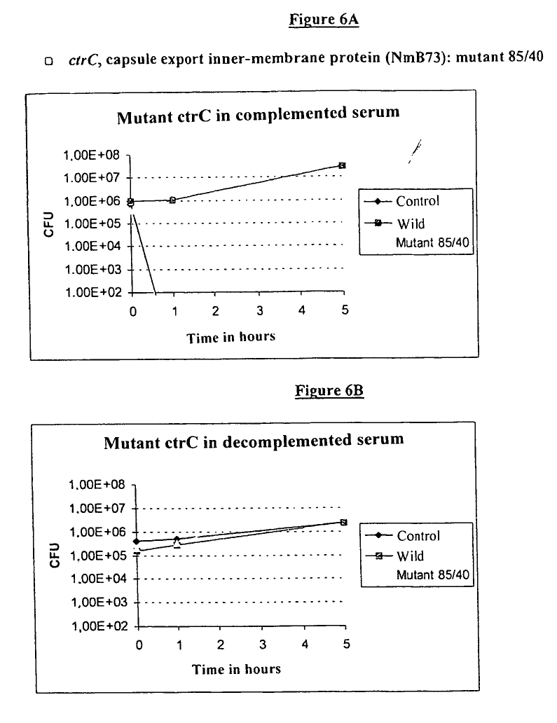

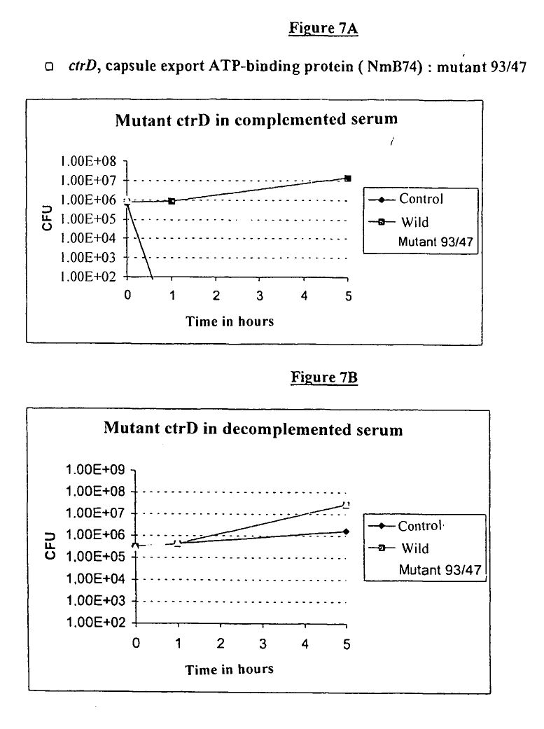

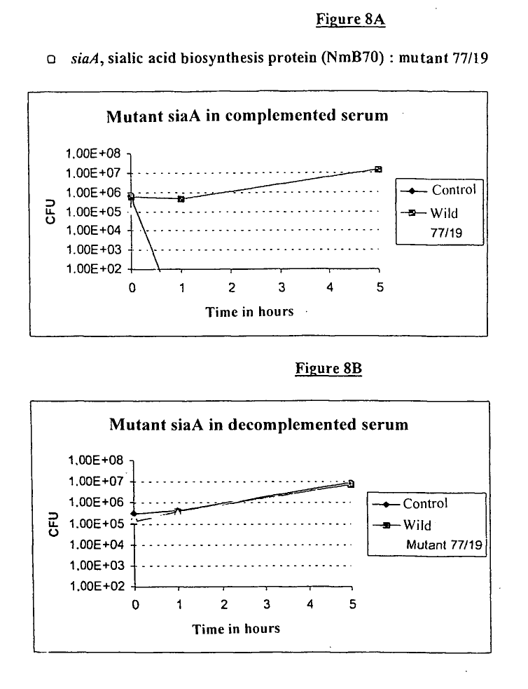

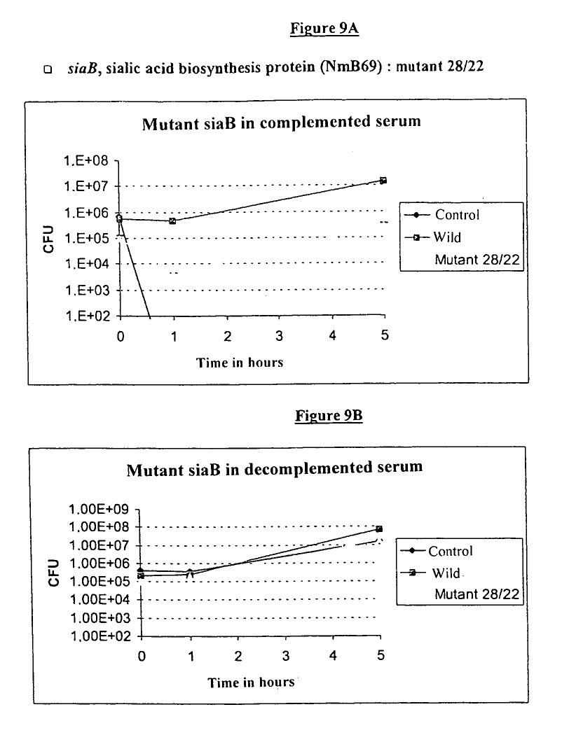

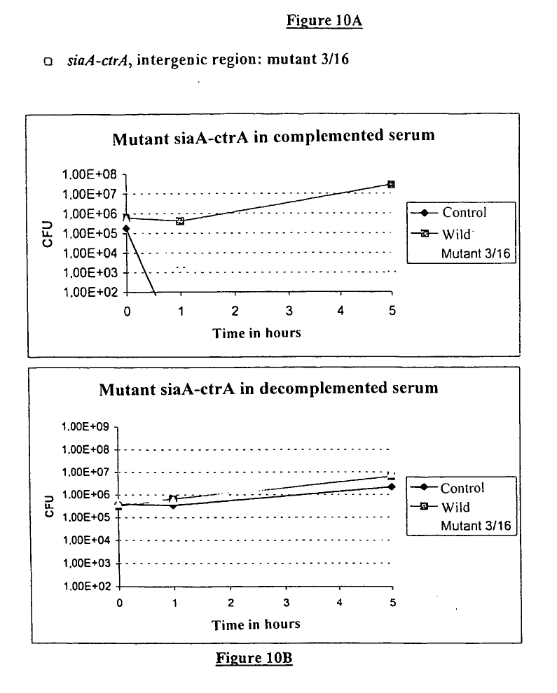

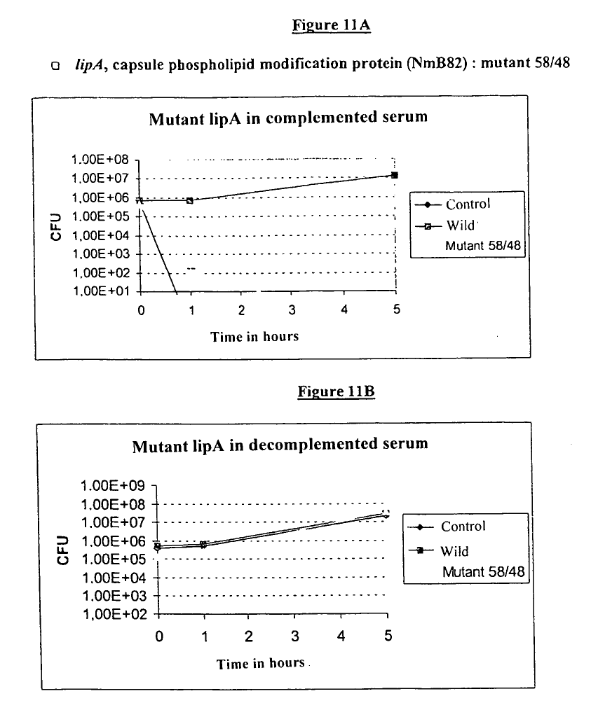

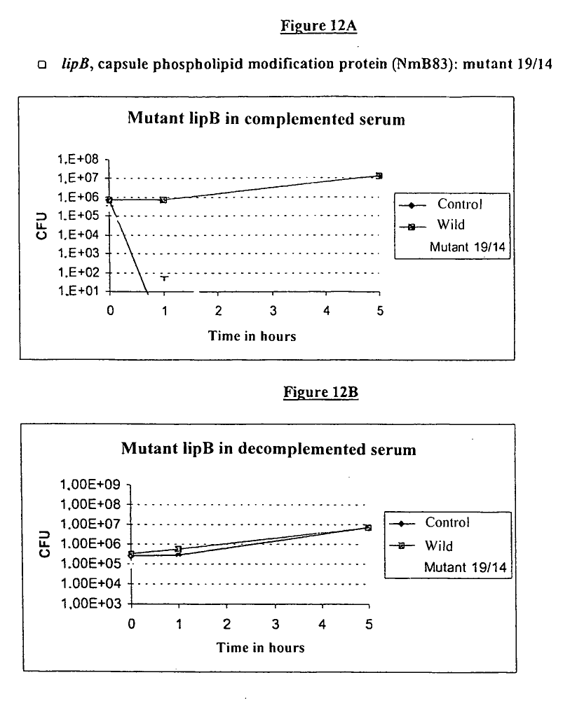

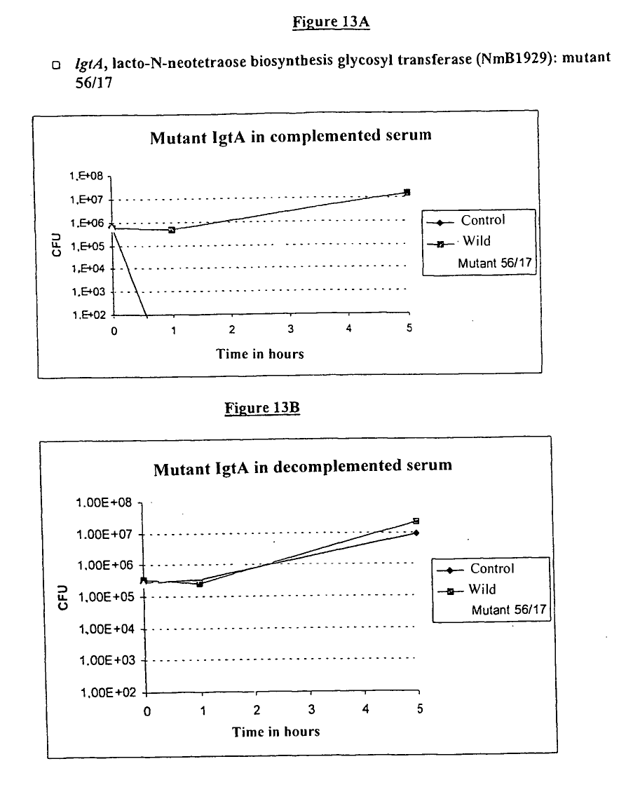

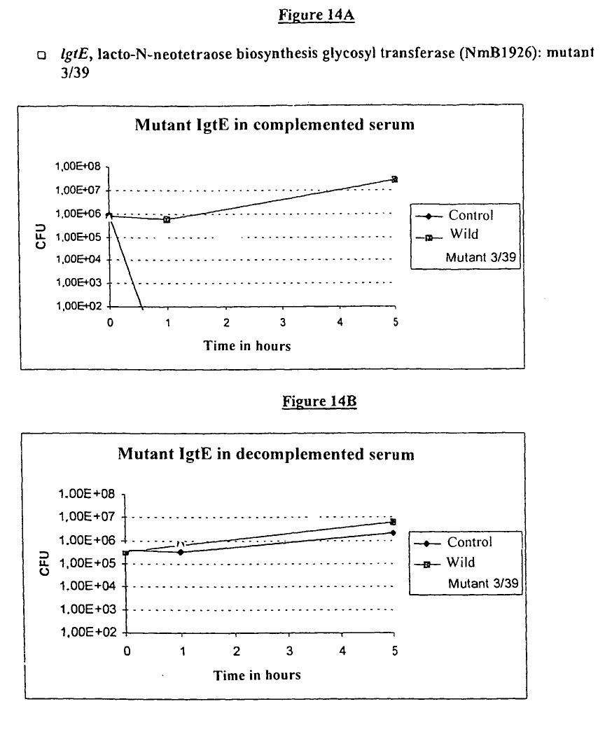

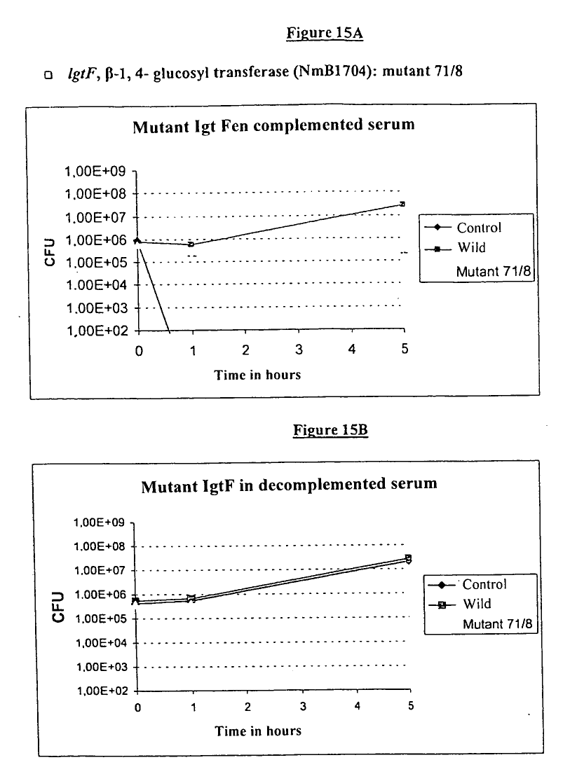

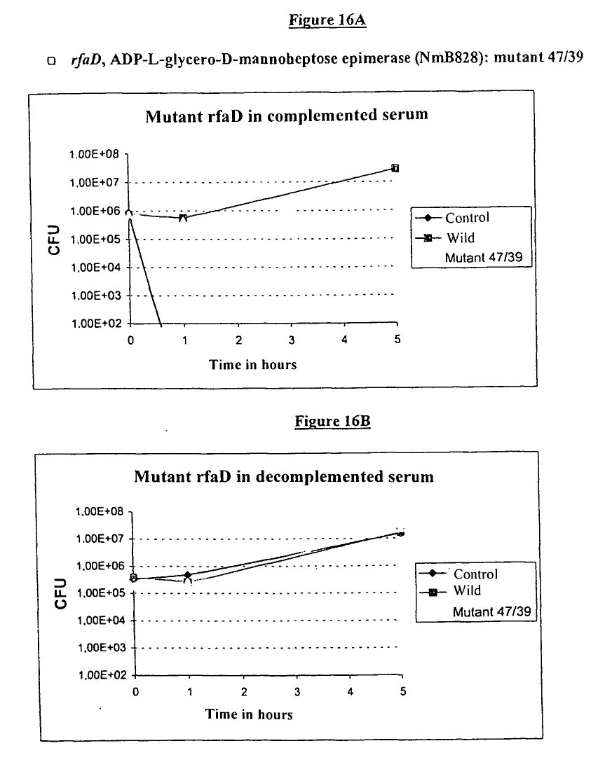

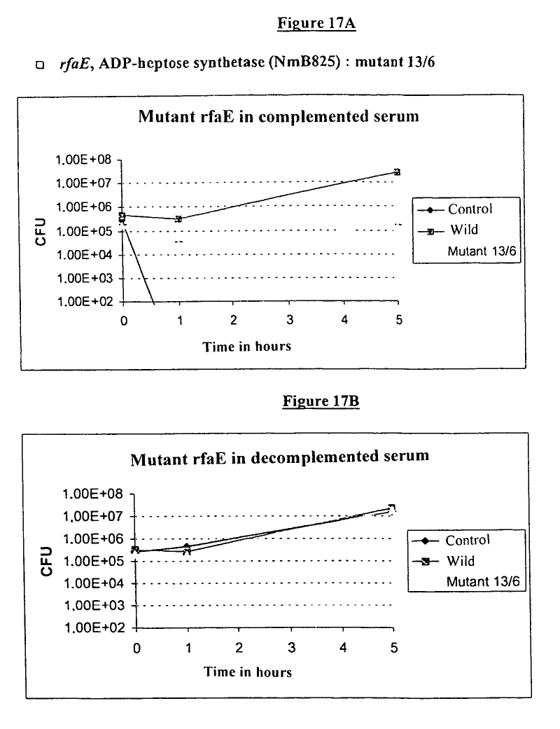

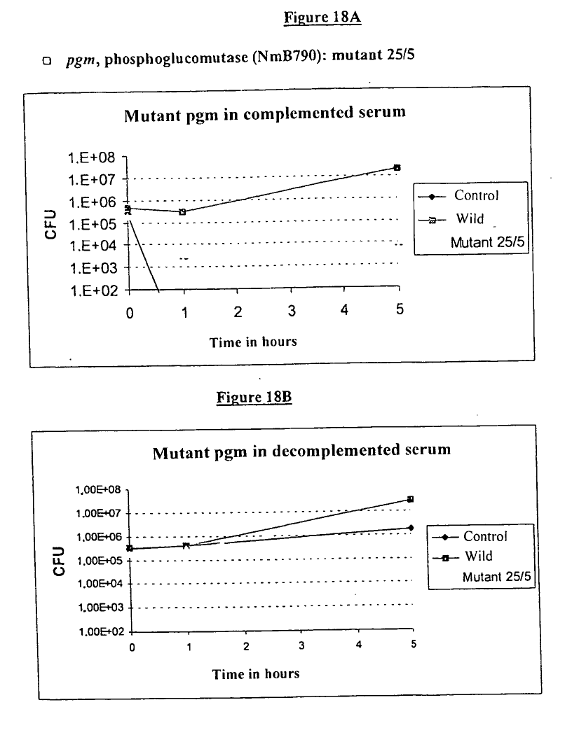

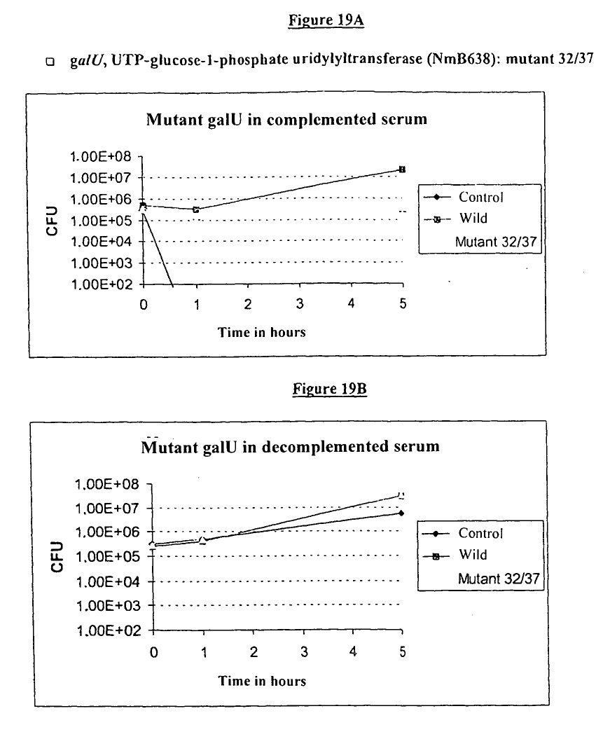

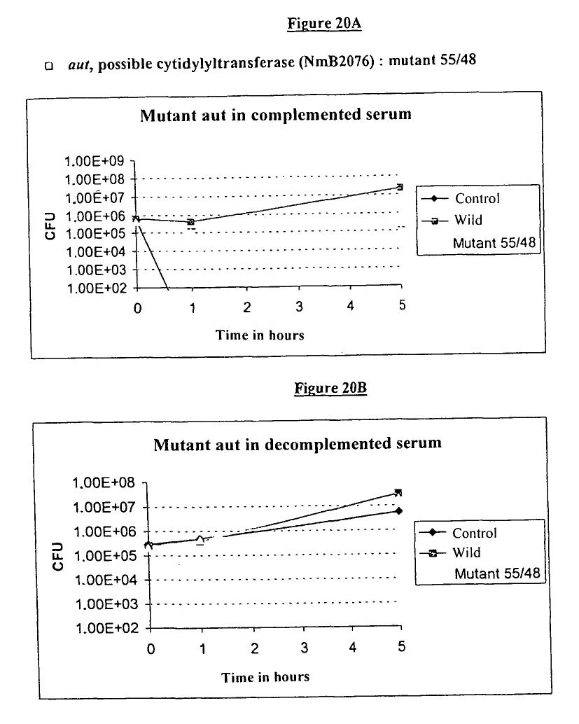

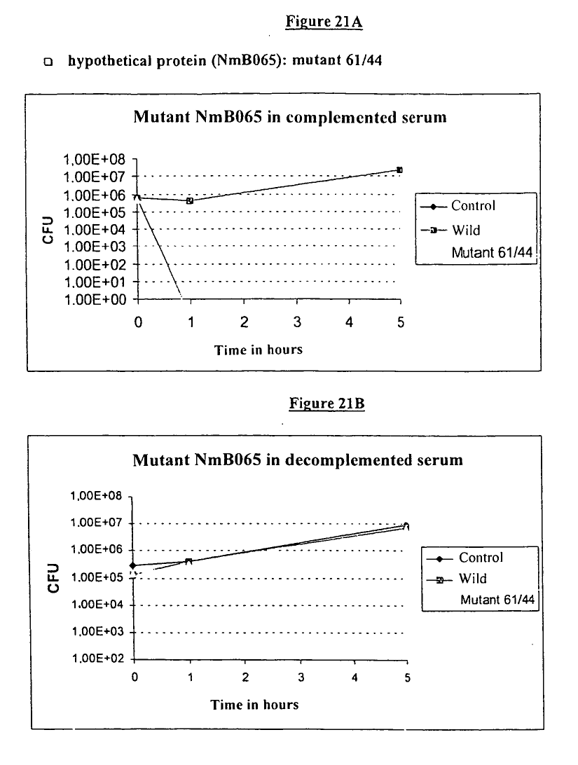

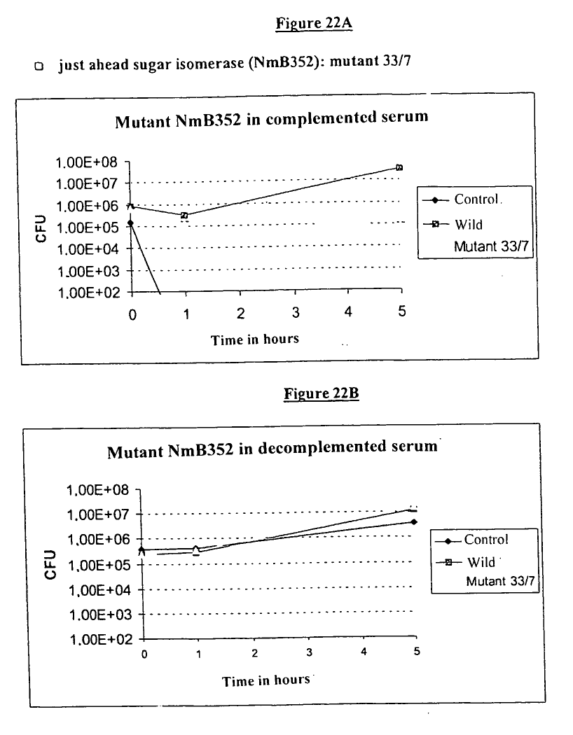

Other characteristics and advantages of the invention are given in the examples which follow and with reference to FIGS. 1 to 25 which represent:

FIG. 1, the list of genes presenting in the 2 sequenced strains of Nm more than 70% similarity on a proteinic base,

FIG. 2A, the list of genes for which there exists a mutant in the bank,

FIG. 2B the list of mutants classified into 96 pools of 48 mutants,

FIG. 2C, the list of essential genes of Nm without mutants in the bank and

FIG. 2D, the list of essential genes of Nm having a homology of 40, 60, 80% with an E. coli K12 gene,

FIG. 3, the list of the mutants altered in growth in the serum,

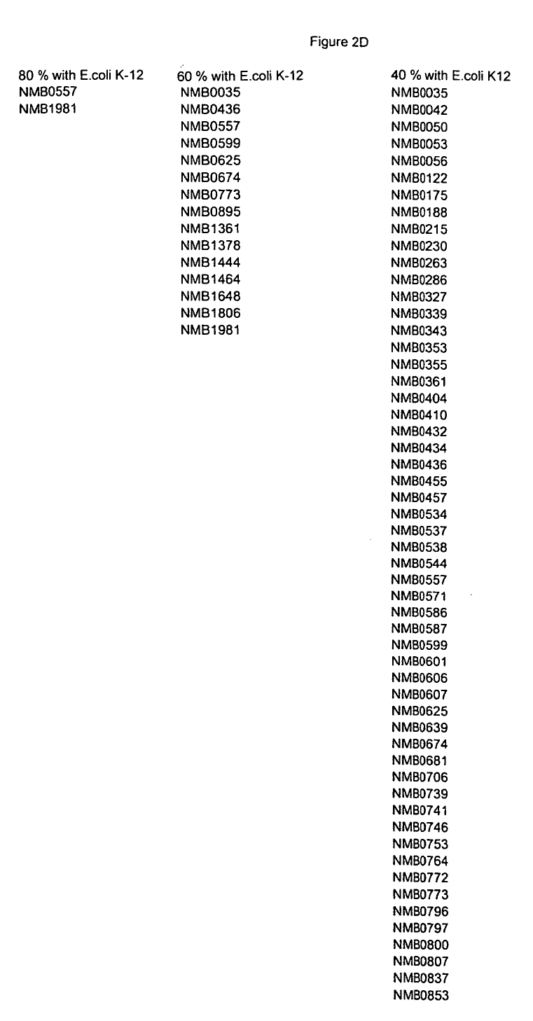

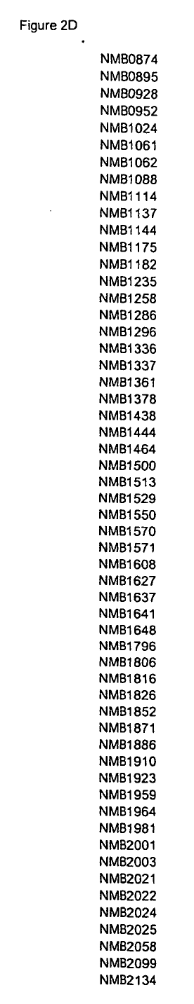

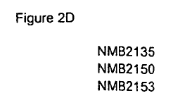

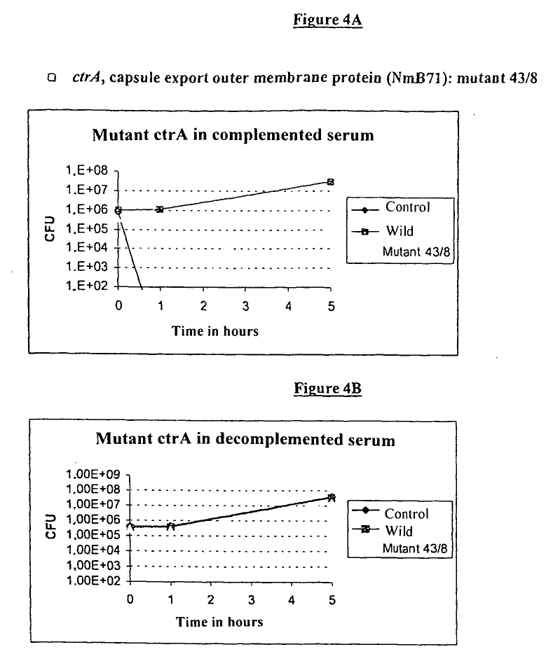

FIGS. 4 to 24, the growth curves of the mutants of the figure in the complemented serum and the decomplemented serum, and

FIG. 25, the number of colony forming units, as a function of the time, with a wild strain of Nm (WT), a Pil⁻ strain and a Nml1110⁻ strain.

Construction of a Bank of Mutants of Nm 8013

1. A bank of mutants is built from the N. meningitidis 8013 strain of serogroup C, operating according to the technique described by Pelicic et al, Journal of Bacteriology, 2000, 182: 5391-5398. A sequenced bank of 4547 mutants is obtained.

Statistically 80% of the insertions are in open reading frames since it concerns the % of coding regions in the genome of the 2 sequenced strains, namely Z2491, strain of serogroup A sequenced by the Sanger Center, and MC58, strain of serogroup B sequenced by TIGR. Therefore there are approximately 3600 mutants in open reading frames and in most cases, several insertions per gene. Taking into account the size of the genome, the mutagenesis thus concerns 93% of the mutagenizable genes.

The statistic formula allowing calculation of the probability (P) that a gene is mutated is the following:
P=1−e^{-n/P}
n: number of mutants in genes (71% of the mutants are in genes as determined through sequencing in the insertion sites),
p: number of mutagenizable (non-essential) genes

The second number can only be estimated. But according to studies of bacteria better characterized than Neisseria meningitidis, it is reasonable to estimate that 350 genes are essential to the survival of the bacteria. Consequently, there would be 1470 non-essential genes in the meningococcus, 88% of which should be mutated in the bank.

2. All of the insertions of this bank are sequenced according to the technique used for the sequencing of insertions, already described and published (Prod'hom et al. 1998. FEMS Microbiol Left. 1858: 75-81). This technique uses a specific primer for the known sequence, in this case the transposon, and a second specific primer of a synthetic linker ligated to the reduced genomic DNA. The use of AmpliTaq Gold polymerase Perkin-Elmer is important for minimizing a non-specific hybridization of the primers.

The examples given below illustrate the following results:
3801 insertions (83.6%) of the 4548 mutants have been sequenced,
3221 insertions have been able to be placed using the genomes of MC58 or Z2491,
580 insertions (15.3%) are in repeated or specific regions of the strain used for the mutageneses.

Determination of the Essential Genes.

An essential gene can be present only in a single strain. Any gene present in the two strains, the genome of which has been sequenced and for which a mutant does not exist in the bank of the invention is thus considered to be essential.

The genes present in the two strains are given in FIG. 1. The nomenclature used is that of the strain Z2491 (sequenced by Sanger). The list given in FIG. 1 was obtained by performing a TblastN of each reading frame of Z2491 in MC58, then keeping all the frames of Z2491 which had a homology percentage greater than 70%. The genes possessing a mutation are identified in bold type.

The list of the genes for which a mutant is present in the bank is represented in FIG. 2A. The list of differential genes, i.e. those present in FIG. 1 and not in FIG. 2A, is high in essential genes. The genes in which the mutants are found in the transposases are underlined and in bold. This list of differential genes includes genes which are homologous in other Gram-negative pathogenic bacteria, such as enterobacteria, Pseudomonas, Acinetobacter, or even certain Gram-positive bacteria. FIG. 2C gives the list of the essential genes of Neisseria meningitidis having a homology of 40, 60, 80% with a gene of E. coli K12. These genes constitute targets for developing broad spectrum antibiotics against these Gram-negative bacteria and broader spectrum antibiotics when these genes are homologous to certain genes of Gram-positive bacteria.

Screening of the Bank for Different Phenotypes.

For the screening, knowledge of the sequence of each insertion is applied. For this, the mutants are organized into pools of 48. For each mutant, the insertion sites are amplified using suitable oligonucleotides. Each amplification product is deposited on a nylon membrane. The pools of 48 mutants are then placed in the conditions for which mutants are sought. The total DNA is prepared using bacteria obtained from each output pool and an amplification is carried out using oligonucleotides which served to amplify the 48 insertion sites. The amplification product then serves to hybridize the membranes which correspond to each pool. The mutants for which no amplification is detected are mutants for the phenotype considered.

Search for Mutants Important for Growth in Serum

As mentioned above, N. meningitidis is a bacteria having extracellular multiplication perfectly suited to this compartment. The invention therefore related to identifying in an exhaustive way the attributes and the genes required for this growth.

1-Isolation of the Strains

The wild strain 2C43 wt (positive control) and Z5463 CPS- (non-capsulated strain, negative control) are isolated on a GCB box (agar 5 g/1); the mutants produced from the strain 8013 are isolated on a GCB box+Kanamycin 100 pg/pl.

The culture is carried out over 14-18 h, at 37° C., in 5% CO2.

2-Serum

The complemented human serum is stored at −80° C. After heating for 30 min. at 56° C., the serum is decomplemented. Growth is produced for the controls and the mutants with systematically complemented and decomplemented serum.

Each mutant is tested with a positive and a negative control to compare the growth curves produced on different days.

3-Inoculum

1 dose of well-isolated colonies is collected and dissociated in 5 ml of RPMI (GIBCO: RPMI 1640 medium with glutamax I; previously placed for 5-10 min. in ambient temperature before inoculation, to protect the bacteria from rapid variations at temperature). The mass of bacteria is taken up using a P1000, then vortexed. The preculture is subjected to stirring at 37° C. for 2 h. The OD is then measured at 600 nm (the white control being RMPI) and the inoculum is returned to 0.1 in RPMI (previously placed for 5-10 min. at ambient temperature).

4-Growth Medium 98 μl of serum and 292 μl of RPMI (25% serum, 75% RMPI) is deposited per well and left for 5 min. at ambient temperature before inoculation. 400 μl of water is introduced into the optionally empty wells.

5-Inoculation After stirring, 10 μl of inoculum adjusted to 0.1 of OD is collected, and it is deposited in a well containing growth medium, then mixed using a P1000. The well is placed in an oven at 37° C., in 5% C0₂. The inoculum is analyzed at T0 and the bacterial growth at various times, by plating 50 μl of different dilutions on GCB boxes.

6-Sampling

Suspension takes place again (with a P1000) before sampling at 0 h, 1 h, 5 h post inoculation. 20 μl of inoculated culture medium is taken which is placed in 180 μl of RPMI (D1; tube 1.5 ml, previously placed at ambient temperature for 10 min., before sampling, in order to avoid a large difference in temperature). The mixture is vortexed.

7-Dilutions

The tube DI is vortexed, then 50 μl of D1 is sampled which is added to 450 μl of RPMI (D2; 2 ml tube, previously placed at ambient temperature for 10 min.). Between each dilution stage vortexing is carried out and the cone is changed. Dilutions are carried out up to the dilution D4 for the time T0, D3 for the time T1, and D5 for the time T5.

8-Inoculation

The inoculation takes place on a GCB box for the controls, and GCB+kanamycin 100 μg/μl for the mutants. Vortexing is carried out, then 50 μl is taken from each dilution, followed by incubating upside down in an oven at 37° C., in 5% C02, for 14-18 h, before counting the colonies. D4, 3 are inoculated for the time TO; D0, 1, 2, 3 for the time T1; D5, 4, 3, 2, 1 for the time T5.

9-Genes of Nm Allowing Growth in Serum: Counting the Surviving Bacteria in Serum as a Function of Time

A growth curve representing the number of bacteria surviving in the serum as a function of time was drawn up for each of the clones (log₁₀ CFU as a function of incubation time in hours).

Two control strains were included each time in the test: the wild strain corresponding to a strain of Neisseria meningitidis, serogroup C and a control strain corresponding to Neisseria meningitidis, serogroup A without capsule. For each gene a single mutant is represented.

The results are given in FIGS. 4 to 24, which represent the growth curves of the mutants of FIG. 3 in the complemented serum and the decomplemented serum.

Identification of the Adhesins for Endothelial Cells.

The important adhesins for interaction on endothelial cells can be used to allow the opening of the blood-brain barrier and to allow medicaments to pass into the brain.

HUVEC cells at confluence are inoculated in 24-well cell culture microplates at a density of 10⁵/well. The cells are washed the following day in 10% serum/RPMI, and are incubated for 2 h at 37° C. At the same time, the bacteria are resuspended in the same medium at a OD₅₅₀ of 0.1 to 0.01 and incubated for 2 h at 37° C. The suspension of bacteria is used to infect the cells for 30 min at 37° C.

The infection then continues for 4-5 h with the cells being washed each hour.

The percentage of adhesion of each mutant compared to the wild strain is then measured. There are two types of mutants: linked mutants, which are important for piliation and mutants not linked to the pili.

The results are given in Tables 1 and 2 below:

Table 1 relates to mutants in 4 genes: these mutants are piliated, but defective in adhesion (they are capable of crossing the blood-brain barrier and are used for the development of vaccines).

TABLE 1

| Gene (nomenclature | | |
|---|---|---|
| Z2491) | function | % adhesion |
| Nm 1110 | conserved protein | 1 |
| Nm 1111 | conserved protein | 25 |
| Nm 1372 | dehydrogenated | 2 |
| | inosine-5′- | |
| | monophosphate | |
| Nm 1892 | conserved protein | 5 |

TABLE 2

| Gene | | |
|---|---|---|
| nomenclature Z2491 | Function | Piliation |
| Nm264 | pilE, pilin | — |
| Nm650 | pilQ, pilus secretin | — |
| Nm654 | pilM, pilus-assembly | — |
| | protein | |
| Nm1106 | conserved protein | +/− |
| Nm1107 | membrane protein | — |
| Nm1108 | membrane protein | — |
| Nm1109 | membrane protein | — |
| Nm1523 | lipoprotein | — |
| Nm2155 | pilus-assembly protein | — |
| Nm2156 | pilD, prepilin peptidase | — |
| Nm2159 | pilus-assembly protein | — |

FIG. 25 shows the number of colony forming units, as a function of time, with a strain of wild Nm (WT), a pilD- strain and an Nm1110⁻strain. The results obtained show that Nm1110 is necessary for adhesion.

1. Exhaustive method for detecting pathogenic bacteria genes, in particular Nm, expressing a desired phenotype, characterized in that:
a bank of mutants generated from a given bacterial strain is used so that at least 70% of the non-essential genes, and in particular at least 80%, or even more than 90%, are mutagenized by inserting a transposon in a reading frame,
the mutants are then brought into contact, either individually, or in pools, with an environment, such as a medium, an animal or cells, capable of interacting with the mutant bacteria expressing the desired phenotype,
when pools are used, the bacteria not having reacted with the desired phenotype are recovered,
the mutated genes of these bacteria are identified and their involvement in said phenotype is verified.

2. Method according to claim 1, characterized in that, in the contact stage, the mutants of the bank are passed through serum.

3. Method according to claim 1, characterized in that, in the contact stage, the mutants of the bank are passed over endothelial cells.

4. Isolated Nm genes, which give a bacteria the ability to grow or to interact with a given environment, such as serum, an in vivo animal model, cells, characterized in that they can be obtained by the method according to claim 1.

5. Nm genes according to claim 4, characterized in that they are implicated in the growth of the bacteria in serum and are chosen from those in FIG. 3.

6. Nm genes according to claim 5, characterized in that they are chosen from the genes NmB 352, NmB 065, NmB 2076, NmB 638, NmB828, NmB 825 and NmB 790.

7. Application of the genes selected according to the method of claim 2, as anti-pathogenicity targets, which consists in inhibiting Nm growth in vivo in the serum.

8. Application of the genes selected according to the method of claim 3, for the screening and manufacture of medicaments allowing the opening of the blood-brain barrier to therapeutic ingredients such as medicaments for Parkinson's Disease, Alzheimer's disease, antimitotics, medicaments for multiple sclerosis, antivirals, antimycotics and antibiotics.

9. Application of the essential genes of Nm as targets for developing broad spectrum antibiotics against Gram-negative bacteria when the corresponding protein has a homology of at least 40%, or even 80% with a protein of E. coli.

10. Nm genes according to claim 4, characterized in that they are involved in the interaction with endothelial cells.

11. Nm genes according to claim 10, characterized in that they are chosen from the genes of Tables 1 and 2, especially from NmA 1110, NmA 1111, NmA 1892, NmA 1107, NmA 1108, NmA 1109, and NmA 1523.

12. Application of the Nm genes according to claim 11, in particular of Nm 1110, for the development of vaccines.
